(19) United States
(12) Patent Application Publication
(10) Pub. No.: US 2008/0195033 A1
(43) Pub. Date: Aug. 14, 2008

(54) Method of Delivery of Therapeutic Metal Ions, Alloys and Salts

(75) Inventors: Roy Eagleson, London (CA); Robert Goldsmith, San Francisco, CA (US); Michael J. Rieder, London (CA); Ian R. Marshall, Mississauga (CA); Jan Greenhalgh, Burlington (CA)

Correspondence Address: LANG MICHENER LLP, Brookfield Place, SUITE 2500, 181 BAY STREET, TORONTO, ON, M5J 2T7, omitted

(73) Assignee: INTERNATIONAL BIO-THERAPEUTIC RESEARCH INC., Mississauga, ON (CA)

(21) Appl. No.: 11/720,074

(22) PCT Filed: Nov. 23, 2005
(86) PCT No.: PCT/CA2005/001777

§ 371 Date: Mar. 3, 2008

Related U.S. Application Data
(60) Provisional application No. 60/629,975, filed on Nov. 23, 2004.

(51) Int. Cl. A61N 1/30; A61K 33/38; A61K 9/12; A61K 9/70; A61K 9/00; A61K 9/48; A61M 5/20; A01G 7/04; A61M 11/00; A61P 31/12; A61P 31/10; A61P 31/04; A61P 35/04

(52) U.S. Cl. 604/21; 424/618; 424/43; 424/449; 424/400; 424/451; 604/131; 128/200.23; 47/1.3

(57) ABSTRACT

A method for treating a bacterial, viral, fungal, or vector-induced disease state. A therapeutically effective dose of a metal substance is delivered to the body of a potentially infected organism using a delivery methodology selected from the group consisting of syringes, auto-injectors, pricking devices, buccal embedding, transdermal patches, needle transdermal patches, aerosol inhalers, ingestible dissolvable capsules, encapsulated boluses, needle encapsulated boluses, and electrode catheterization methodologies. The metal substance is selected from the group consisting of silver, gold, copper, zinc, selenium, platinum, and their ions, alloys, salts, and combinations thereof. Preferably, an electrical current is introduced substantially in the course of utilizing the delivery methodology. The electrical current is preferably substantially varied over time, and is still more preferably a reversing electrical current.

### FIELD OF THE INVENTION

This invention relates to methods for the treatment of bacterial, viral, fungal and vector pathogenic states in humans, animals, and plants, and to improvements thereto.

### BACKGROUND OF THE INVENTION

In the human immune system, the body's resistance to disease takes two forms: “nonspecific resistance” and “specific resistance”. Nonspecific resistance exists in all humans and virtually all other species and generally is thought to offer some protection against all parasites. Nonspecific resistance involves species and population immunities and such mechanical and chemical barriers as the skin surface, mucus secretions, stomach acid, lysosyme and interferon. Phagocytosis is a nonspecific mechanism in which macrophages (or large white blood cells) and other phagocytes engulf and destroy microorganisms. Fever and inflammation are other forms of nonspecific resistance. In another form of nonspecific resistance, if Class I MHC proteins are absent from the surface of a cell or have a reduced presence (as in cancer cells or virus-infected cells), the body's Natural Killer (NK) cells will damage those cell membranes and induce lysis.

Conversely, specific resistance develops from the response by the body's immune system to substances called antigens. Antigens are large, complex molecules that the host body interprets as ‘non-self’. Proteins, polysaccharides, and an enormous list of substances containing these molecules are antigenic. A small part of the antigen called the “antigenic determinant” performs the actual stimulation of the immune system. A person's own chemical substances are non-antigenic because they are interpreted as ‘self’.

The actual immune response may originate with the entry of one or more antigens into a host body and their penetration into the lymphatic or cardiovascular system. Here, possibly according to a nonspecific response, macrophages and other phagocytic cells may phagocytize the antigens, and break them down so as to release the antigenic determinants or epitopes. Thereafter, possibly to initiate a specific response, the macrophages may display the epitopes on their surface and transport them to the lymphoid organs, where the epitopes might be presented to waiting T and B-lymphocytes. Another important transporter might be the dendritic cells—cells with long finger-like extensions that form lacy networks in virtually all tissues, and that are capable of phagocytizing infected cells nearby. Phagocytosis and transportation of epitopes are extremely important, because research evidence indicates that unprocessed antigens stimulate the immune system poorly.

It has become apparent that “specific resistance” is a phenomenon that may very well have potentially broader implications, including organ transplantation, allergic reactions and resistance to cancer.

The immune system may originate with bone marrow cells that undergo differentiation to form B-lymphocytes and T-lymphocytes. These cells comprise the tissue of the spleen, lymph nodes and other lymphoid organs, and they are the major underpinnings of the immune system.

When T-lymphocytes are stimulated by epitopes or antigenic determinants that are presented to them by macrophages or other phagocytes, the T-lymphocytes may leave the immune system as ‘cytotoxic cells’ and travel to the infection site. At the infection site, the cytotoxic T-lymphocytes may kill the infecting organisms in a process that may generally be referred to as ‘cell-mediated immunity’. Thereafter, memory T-lymphocytes may remain in the tissue to provide long-lasting protection.

A second aspect of “specific resistance” is antibody-mediated, or humoral, immunity. In this case, B-lymphocytes may be stimulated to form antibody-producing cells called plasma cells. Antibodies may be formed in the lymph nodes and, typically, are protein molecules composed of light and heavy chains of amino acids. The antibodies may enter the circulation system so as to reach the infection site, where they may react with, and neutralize, micro-organisms by various mechanisms. Five types of antibodies are generally recognized, each with its own function and structure.

Cell-mediated immunity may result in activated T-lymphocytes that may be particularly well suited for direct interaction with eukaryotic pathogens, as well as antigen-marked cells, such as virus-infected and transplanted cells. Sometimes called cellular immunity, cell-mediated immunity responds to cells that have been infected with pathogens such as viruses, rickettsiae and certain bacteria, including M tuberculosis, as well as, protozoa and fungi. Together with cytotoxic T-lymphocytes, the antibodies impart “specific resistance” during times of disease, and they remain in the body for long-lasting resistance.

Humans, animals and plants may 'succumb to infections that are bacterial, viral, fungal and vector induced, rendering their body defense mechanisms compromised and sometimes, leading to death. For example, such disease and/or pathogen-induced states may include the following: chagas, dengue, leishmania, encephalitis, rickettsia, candida, tuberculosis, various pneumonias, septicemia, dysentary, polio, measles, chicken pox, small pox, mumps, ebola, HIV, malaria, eye infections, macular degeneration, skin cancers, nasal pharyngeal cancer, and breast and prostate cancer and HPV. By way of further examples, autoimmune diseases may include the following: diabetes, thyroid disorders, arthritis, transplant rejections, and others. By way of still further examples, disease states in animals may include the following: hoof and mouth disease, leishmania, pig cholera, distemper, panleukopenia and feline immunodeficiency disease, as well as others.

What may be needed, therefore, is a new and inventive method to treat any one or more of the above disease states.

At this point, it may also be worthwhile to discuss HIV, or the human immunodeficiency virus, in greater detail. The human immunodeficiency virus has become one of the largest leading causes of death among humans, next to malaria and tuberculosis. Past work with antiviral drugs has thus far failed to provide an effective treatment for HIV infected patients, creating resistance after repeated use, and problems with one time dosages of Nevaripine at time of delivery. Notably, as well, it should be remembered that some of the oral therapies that are presently available and most effective—such as, for example, protease inhibitors used in combination with other drugs—have been shown to be quite toxic, producing many side effects, enough so that some patients are unable to tolerate such therapies. Even where they are able to-tolerate the treatment, however, HIV patients may yet die from infections with secondary opportunists, such as tuberculosis, because of a defeated immune system created by the human immunodeficiency virus' destruction of the immune system. While malaria still remains one of the number one killers in the world, AIDS patients fare poorly when dealing with such vector-induced organisms.

There has been prior art evidence to suggest that HIV infection involves dynamic viral turnover. Moreover, it has generally been thought that control of infectious viral burden in terms of number of circulating viral particles (viral load) might delay or reverse progression of HIV infection to full blown AIDS. This thinking has led to the development of many anti-retroviral drugs that have aimed to reduce viral load in people with HIV infection and AIDS. The promise of drug therapy for people with HIV infection and AIDS has not, however, been completely realized. Among other things, the initial hope that antiretroviral therapy could be withdrawn over time has not been borne out.

For each of the aforesaid reasons, among others, it may be worthwhile to discuss the mechanisms of viral infections, and specifically the background of the human immunodeficiency virus, in relationship with the pandemic world medical situation, as well as various improvements that may be associated with the treatment of viral infections.

Viruses may generally be said to be dependent upon living organisms. For a virus to live and reproduce, it must have a host cell. Viruses may be of many different sizes, shapes, and configurations. Viruses or virions are generally comprised of a viral core that is made up of nucleic acids and that carries the viral genes, as well as fatty acids and proteins that surround the core. Viruses are generally thought to attack host cells by causing, at least, the virus' nucleic acid to enter the cell. The virus then takes over the cell's metabolic machinery, and uses this machinery to make many copies of itself, thus producing many new virions. In the case of the human immunodeficiency virus, the virions are released from the cell by lysing, thereby destroying the cell. Many of these virions are able to go on to infect other host cells, each which may eventually be killed.

One of the reasons that the human immunodeficiency virus is extremely dangerous may be because HIV targets a specific type of T-lymphocyte (or T-cell), and eventually produces so many virions to attack these T-cells that the body cannot make T-lymphocytes fast enough to replace those destroyed by HIV. The specific T-cell that is targeted by HIV is the T4 helper lymphocyte. When an HIV virion finds a T4 cell, it is generally believed that it may attempt to penetrate the cell wall and gain access to the T4 cell's nucleus. After attachment and injection into the cell, the virus is able to enter into the cell's nucleus and splice itself into one of the T4 cell's chromosomes. At that point the T4 cell will be infected with HIV. Thereafter, the T4 cell may begin to reproduce copies of the human immunodeficiency virus, or virions. Thousands of virions are produced within one T4 cell wall until it eventually lyses and destroys the cell. The copies of the infecting human immunodeficiency virus that are released from the destroyed T4 cell may very likely, thereafter, go on to infect other T4 cells. Since an infected T4 cell produces copies of the human immunodeficiency virus faster than humans can produce T4 cells, eventually the immune system of the infected person is overrun and unable to fight off infection. This inability to stave off infection may largely be due to the presence of too few T4 cells remaining in the host to create an adequate immune response to invading agents. It is generally these secondary opportunistic diseases which eventually lead to the death of a patient from HIV.

It may be worthwhile to note, at this point, that it is generally thought (though not essential to the working of the present invention) that all biological life forms have a negative charge. Naturally, positive electrical energy will be highly attracted to any such negative polarization. If positive electrical energy is introduced directly into an animal - such as, for example, into a human that may be standing too near a tree as it is struck by lightning—this introduction of positive electrical energy might be fatal. This electrical phenomenon may occur in both single and multi-celled organisms, and while the healthy individual cells comprising such organisms may have a slightly positive or a slightly negative polarity, it is generally thought (though, once again, not central to the working of the present invention) that an overall substantially neutral electromagnetic field surrounds each such healthy cell. In any case, though, polarized positive cations are not normally thought to be attracted to healthy individual cells.

In contrast, the HIV virion may be generally comprised of a core having nucleic acids, protein-like substances and RNA. One portion of the viral core may be generally thought to have a slight, but distinct, positive polarity, with another portion of the viral core for HIV (and for other viruses and bacteria) being thought to having a slight, but likewise distinct, negative polarity.

What may be needed, therefore, is a novel and inventive method of treating viral disease states that, in one of its embodiments, might capitalize upon this apparently marked polarity of the viral and bacterial cores.

In this discussion of the background of the invention, it is appropriate to note that the use of various metals in anti-infective therapies has been known throughout ages. The unique medicinal properties of silver, gold and copper have been recognized throughout human history.

For example, the therapeutic power of silver has been investigated and/or utilized in Ayurvedic medicine, in Chinese medicine, in homeopathic medicine and in traditional medicine, as well as in the somewhat more esoteric field of gem therapy. Additionally, the Phoenicians, for example, used silver vessels in the hope of keeping water, wine and vinegar pure during long voyages. Similarly, American pioneers put silver and copper coins in their water barrels with the aim of keeping it clean. In fact, the phrase ‘born with a silver spoon in his mouth’ may have had its genesis in an observation that was made in the early 18^{th} century that babies fed with silver spoons were thought to be healthier than those fed with spoons made from other metals. In the prior art, silver may have been used both intravenously and intramuscularly, and as a throat gargle, douche, orally, topically, and as eye drops.

The term “oligodynamic action” (meaning “small power action”) expresses the activity of heavy metals—such as, for example, mercury, silver, and copper—on microorganisms. These elements are called heavy metals because of their large atomic weights and complex electron configurations.

Mercury is a traditional heavy metal antiseptic, with mercuric chloride having been used for centuries by the Greeks and Romans in the treatment of skin diseases. In some of its more recent and various forms, mercury has previously been combined with various carrier compounds so as to be less toxic when applied to the skin, especially after surgical incisions. Other mercury derivatives have previously been used as preservatives in vaccines.

Copper has previously been known to be active against chlorophyll-containing organisms and is a potent inhibitor of algae. In the form of copper sulfate, copper has been incorporated into algicides which have been used in swimming pools and municipal water supplies. Copper sulfate has also previously been mixed with lime to form a bluish-white mixture that has been used since the late 1800s to control the growth of various fungi.

Silver, in the form of silver nitrate, has likewise previous been known to be useful as an antiseptic and as a disinfectant. For example, drops of a diluted silver nitrate solution have heretofore been placed in the eyes of newborns to protect against infection by Neisseria Gonorrhea—a gram negative diplococcus bacteria that can cause blindness if contracted by newborns during passage through the birth canal. This treatment was first used, in the late 1800s, to prevent gonococcal eye infection, and many jurisdictions still utilize this method. Although effective, this therapy has largely been superseded by other therapies that have generally been perceived to be less irritating.

Similarly, the use of silver ions has been shown to be quite effective (both in vitro and in vivo) in inactivating many species of bacteria, fungi and viruses, including the herpes simplex virus. Various silver compounds have also been used in the past to treat suturing threads. Additionally, the use of silver ions has been shown to have some role in suspending mitosis in fibrosarcoma cells. Further, anecdotal evidence appears to suggest that silver ions may help to de-differentiate fibroblast cells and keep them “uncommitted”, but able and ready for further differentiation.

In recent years, silver therapy involving the oral administration of colloidal silver particles has been the subject of significant interest within the medical community and has also gained much trust. Silver salts, such as silver chloride, have been known to appear naturally in human blood serum at concentrations of approximately thirty to eighty parts per billion.

While some in vitro studies have been performed in association with silver ion therapy, silver ion therapy has not been studied extensively, especially in vivo. Notwithstanding this fact, U.S. Pat. No. 4,292,968—the teachings of which are incorporated herein by reference—was issued to Franklin H. Ellis on Oct. 6, 1981 for an ELECTRIC SUPPLY FOR ION THERAPY, and it teaches the use of a power supply in ion therapy to provide direct current to electrodes attached to a patient. Another example, in this regard, can be seen in U.S. Pat. No. 5,470,349—the teachings of which are likewise incorporated herein by reference—that was issued to Bernhard Kleditsch and Gabriel Khazaka on Nov. 28, 1995 for a DEVICE FOR TREATING INFLAMMATORY SKIN CHANGES IN THE INITIAL STAGES AND METHOD FOR USING SAME, wherein it was taught that an electrode and its counter-electrode(s) might be pressed against the skin before the current is passed. In both cases, use of an exceedingly cumbersome device was disclosed, which heretofore has made it rather difficult to achieve patient compliance.

Gold and copper may have been indicated, in the prior art, to have a role in alleviating the pain of inflammation in diseases such as arthritis. Bracelets, pendants, and chains of these metals may heretofore have been “prescribed” by many different cultures and societies for several centuries.

Additionally, formulations containing zinc and/or selenium may likewise have been implicated, in the prior art, as having had a role in arresting the deterioration of, and/or even in reversing, opthalmological disorders such as the macular degeneration and weakening of the retina.

In addition to the clinically documented and other references that may have been touched on hereinabove, there have also been numerous anecdotal references throughout human history that may have related to the use of metals, including as well many different alloys and/or salts thereof, in disease therapy.

What may be needed, therefore, is a method of using metal substances to treat disease states that might, in a new and inventive manner, take advantage of this rich history of anecdotal and clinically tested evidence which would seem to favor their use. Understanding the mechanism of the action of metal ions (such as silver, gold, copper, and others) is not, however, the focus of the invention, but rather it is associated primarily the therapeutic application of new and innovative technologies.

At this point, it may be worthwhile to discuss the potential role of metal substances in preventative medicine. It may be generally well-known, in the prior art, to prevent infection by many viruses through the process of vaccination. This process typically involves the injection of an uninfected patient with a weakened or denatured virus. In response to this injection, the body may create antibodies that are specific to that virus. In other preventative situations, humans may have been known to take antibiotics or vitamins, or to partake of a nutritional diet, so as to support good health. Moreover, individuals have been generally counseled to attend annual physical check-ups with their local doctor and have diagnostic tests performed so that they might be kept apprised of their well-being.

Frequently, however, in the cases of both HIV and those individuals most likely to be affected by a seriously harmful disease state (i.e., those which are mostly present in the Third World and in developing areas), there may either be no vaccination and/or no realistic other medical option for prevention. In such circumstances, not only is there no vaccine for HIV, there may be little else to afford a diagnosis and/or a follow-up treatment. Though there may be some harsh drug regimens and/or treatments for HIV/AIDS patients, these treatments are generally expensive and caustic to the human body. In addition, there may now be emerging proof of resistance associated with the use of any the previously most preferable drugs of choice.

Thus, there has been a long felt need for a treatment that might be used in the case of patients infected with blood-borne pathogens, such as HIV, so as to thereby destroy such pathogens and/or uplift the human immune system.

Accordingly, it is an object of the invention to obviate, mitigate, and/or address one or more of the needs, shortcomings and/or disadvantages associated with the prior art.

### SUMMARY OF THE INVENTION

In accordance with the present invention, there is disclosed a method for treating a disease state in the body of an organism. According to the method, a therapeutically effective dose of a metal substance is delivered to the body of the organism using a delivery methodology that is selected from the group consisting of syringe, auto-injector, and pricking device delivery methodologies, buccal embedding techniques, transdermal patch methodologies, and aerosol inhaler techniques. The metal substance is selected from the group consisting of silver, gold, copper, zinc, selenium, platinum, and their ions, alloys, salts, and combinations thereof.

According to a further aspect of the invention, the method also includes the additional step of introducing an electrical current to the body of the organism substantially in the course of utilizing the delivery methodology.

According to different aspects of the invention, the electrical current may, but not necessarily, be substantially constant, varied over time, and/or intermittent. Where the electrical current is varied over time, it may be varied according to a preprogrammed schedule.

According to one aspect of the invention, the electrical current is preferably, but not necessarily, a reversing electrical current.

According to an aspect of a preferred embodiment of the invention, the therapeutically effective dose of the metal substance, in a colloidal suspension with a pharmaceutically acceptable carrier, may be loaded into a dosage chamber of an auto-injector device. An electrode may preferably, but not necessarily, be formed substantially adjacent to a distal end portion of the dosage chamber. Introduction of the electrical current into the electrode preferably, but not necessarily, facilitates the delivery of the metal substance to the body of the organism.

According to an aspect of another preferred embodiment of the invention, the therapeutically effective dose of the metal substance, in a colloidal suspension with a pharmaceutically acceptable carrier, may preferably, but not necessarily, be embedded in a transdermal patch. Similarly, an electrode formed from the metal substance may preferably, but not necessarily, be embedded in the transdermal patch. Introduction of the electrical current into the electrode preferably, but not necessarily, facilitates the delivery of the therapeutically effective dose of the metal substance from the transdermal patch into the body of the organism.

According to an aspect of a further preferred embodiment of the invention, the transdermal patch may preferably, but not necessarily, be a needle transdermal patch. The electrode may preferably, but not necessarily, be shaped to define one or more needle members that are formed from the metal substance. Preferably, but not necessarily, at least one of the needle members substantially penetrates an outer layer of skin on the body of the organism.

According to an aspect of another preferred embodiment of the invention, substantially particulate portions of the metal substance may be each respectively encapsulated within a pharmaceutically acceptable carrier and loaded, with a propellant, into a reservoir of a canister, which is itself loaded into an aerosol inhaler device. An electrode may preferably, but not necessarily, be provided in the aerosol inhaler device and formed substantially adjacent to a distal end portion of the canister. The particulate portions encapsulated within the pharmaceutically acceptable carrier are preferably delivered into the respiratory system of the organism from a proximal end portion of the canister of the aerosol inhaler device. Introduction of the electrical current into the electrode may preferably, but not necessarily, facilitate spray delivery of the therapeutically effective dose of the metal substance.

In accordance with other embodiments of the present invention, there is also disclosed another method for treating a disease state in the body of an organism. According to these embodiments of the method, a therapeutically effective dose of a metal substance is delivered to the body of the organism using a delivery methodology that is selected from the group consisting of ingestible dissolvable capsule methodologies, encapsulated bolus methodologies, and electrode catheterization methodologies. As with the other methods, the metal substance is selected from the group consisting of silver, gold, copper, zinc, selenium, platinum, and their ions, alloys, salts, and combinations thereof. According to these embodiments of the method, an electrical current is introduced to the body of the organism substantially in the course of utilizing the delivery methodology. According to these embodiments of the method, the electrical current is substantially varied over time and is a reversing electrical current.

According to an aspect of one preferred embodiment of the invention, the therapeutic dose of the metal substance may preferably, but not necessarily, be loaded into a dissolvable capsule. Similarly, the dissolvable capsule may preferably, but not necessarily, be secured to an end portion of an electrically conductive string member that is preferably, but not necessarily, encased in a biocompatible insulating material. The string member is preferably, but not necessarily, in electrical communication with an electrode situated within the dissolvable capsule. The electrode is preferably at least coated with the metal substance. The dissolvable capsule is preferably, but not necessarily, introduced into at least one of the windpipe and the foodpipe of the organism. Introduction of the electrical current into the string member from an external electric current source, and from there into the electrode, preferably, but not necessarily, dissolves the capsule and/or delivers the therapeutically effective dose of the metal substance into at least a respective one of the lungs and the stomach of the organism.

According to an aspect of another preferred embodiment of the invention, a cathode may preferably, but not necessarily, be embedded substantially adjacent to a cationic chamber that is defined within an encapsulated bolus device. Similarly, an anode may preferably, but not necessarily, be embedded substantially adjacent to a separate anionic chamber that is further defined within the encapsulated bolus device. The therapeutically effective dose of the metal substance, in a colloidal suspension with a pharmaceutically acceptable carrier, may preferably, but not necessarily, be embedded in at least one of the anionic chamber and the cationic chamber. The preferable introduction of the electrical current into at least one of the anode and the cathode may preferably, but not necessarily, facilitate the delivery of the therapeutically effective dose of the metal substance from the encapsulated bolus device into the body of the organism.

According to a further aspect of this preferred embodiment of the invention, substantially between about 1 milliamp per minute and about 500 milliamps per minute may preferably, but not necessarily, be introduced to the body of the organism.

According to an aspect of a further preferred embodiment of the invention, an electrical conductor may preferably, but not necessarily, be disposed within a lumen of a catheter. A first electrode may preferably, but not necessarily, extend out of the lumen into the blood stream of the organism. The first electrode is preferably, but not necessarily, in electrical communication with the electrical conductor. A second electrode may preferably, but not necessarily, be placed on the skin of the organism. Alternately, the second electrode may be in direct contact with the blood stream of the organism. The electrical current may be introduced into the electrical conductor, and the first electrode, either from an external electric current source or from an internal battery, so as to preferably, but not necessarily, deliver the therapeutically effective dose of the metal substance into the body of the organism.

According to a further aspect of one such preferred embodiment of the invention, this method may preferably, but not necessarily, be carried out under hydration conditions, with a regimen adapted to substantially hydrate the organism being carried out before and during the method.

According to an aspect of one preferred embodiment according to the invention, the electrical current is preferably, but not necessarily, substantially within the range of between about 0.001 amps and about 0.01 amps. A corresponding electric potential is substantially within the range of between about 0.5 volts and about 3.0 volts.

According to further aspect of this preferred embodiment, the electric potential is substantially in the order of about 1.0 volts, with the electrical current being substantially in the order of about 0.01 amps.

According to an aspect of one preferred embodiment according to the invention, the metal substance may preferably, but not necessarily, comprise silver ions produced by a reversing electrical current. The reversing electrical current is preferably, but not necessarily, a reversing DC current that alternates according to a substantially even duty cycle of about 1 second in the positive direction and about 1 second in the reverse direction. Preferably, the duty cycle continues substantially as aforesaid for a duration of about 15 minutes.

According to an aspect of another preferred embodiment according to the invention, the metal substance may preferably, but not necessarily, comprise silver ions produced by a reversing electrical current. The reversing electrical current is preferably, but not necessarily, a reversing DC current that alternates according to an at least partially asymmetrical duty cycle of about 10 seconds in the positive direction and 1 second in reverse direction. Preferably, the duty cycle continues substantially as aforesaid for a duration of about 15 minutes.

According to an aspect of yet another preferred embodiment according to the invention, the reversing electrical current may preferably, but not necessarily, include variations in cycle length, in electrical current strength, and/or in electrical current duration.

According to aspects of various preferred embodiments according to the invention, the method may be used to treat bacterial, viral, fungal, and/or vector-induced disease states.

According to other aspects of the various preferred embodiments according to the invention, the method may be used to improve plant (e.g., banana plant), animal, and human health.

According to still further aspects of the various preferred embodiments according to the invention, the delivery of the metal substance to the body of the organism may preferably, but not necessarily, be varied according to the species and the body weight of the organism.

According to various preferred embodiments according to the invention, in animals, the method may be used to treat or to preventatively treat hoof and mouth disease, leishmania, pig cholera, distemper, panleukopenia, panleukemia, heartworm disease, Johne's disease, feline immunodeficiency disease, and/or symptoms associated therewith.

According to various preferred embodiments according to the invention, in humans, the method may be used to treat or to preventatively treat chagas, dengue, leishmania, encephalitis, rickettsia, candida, tuberculosis, pneumonia, septisemia, dysentery, polio, measles, chicken pox, small pox, mumps, ebola, malaria, eye infections, macular degeneration, retinal weakening, precursors to cancer, HPV, skin cancers, nasal pharyngeal cancer, breast cancer, prostate cancer, other carcinomas, diabetes, thyroid disorders, arthritis, transplant rejections, other autoimmune disease states, HIV, and/or symptoms associated therewith.

### BRIEF DESCRIPTION OF THE DRAWINGS

The novel features which are believed to be characteristic of a method of delivery of therapeutic metal ions, alloys and salts according to the present invention, as to its method, use, and associated structures and organization, together with further objectives and advantages thereof, will be better understood from the following drawings in which at least one presently preferred embodiment of the invention will now be illustrated by way of example. It is expressly understood, however, that the drawings are not necessarily depicted to scale and are for the purpose of illustration and description only. For these and other reasons, it should be appreciated that the drawings are not intended as a definition of the limits of the invention. In the accompanying drawings:

FIG. 1 is a depiction of a first step in an auto-injector delivery method according to the invention;

FIG. 2 is a depiction of a second step in the auto-injector delivery method of FIG. 1;

FIG. 3 is a depiction of a third step in the auto-injector delivery method of FIG. 1;

FIG. 4 is a depiction of a fourth step in the auto-injector delivery method of FIG. 1;

FIG. 5 is a depiction of an aerosol inhaler delivery method according to the invention;

FIG. 6 is a top view of a reservoir transdermal patch usable in another preferred method according to the invention;

FIG. 7 is cross-sectional view of the reservoir transdermal patch of FIG. 6 taken along sight line 7-7, shown in use;

FIG. 8 is a view similar to FIG. 7 showing a drug-in-adhesive transdermal patch, as used in a further preferred method according to the invention;

FIG. 9 is a top view of a needle transdermal patch usable in another preferred method according to the invention;

FIG. 10 is cross-sectional view of the needle transdermal patch of FIG. 9 taken along sight line 10-10, shown in use;

FIG. 11 is an exploded perspective view of an encapsulated bolus device usable in a further preferred method according to the invention;

FIG. 12 is a top view of a further encapsulated bolus device usable in another preferred method according to the invention;

FIG. 13 is cross-sectional view of the encapsulated bolus device of FIG. 12 taken along sight line 13-13, shown in: use;

FIG. 14 is a view similar to FIG. 13, showing the encapsulated bolus device thereof used in a reversed current configuration;

FIG. 15 is a view, similar to FIG. 13, of a needle encapsulated bolus device, shown in use in a further preferred method according to the invention;

FIG. 16 is a view similar to FIG. 15, showing the needle encapsulated bolus device thereof used in a reversed current configuration;

FIG. 17 is a side view of a dissolvable capsule usable in another preferred method according to the invention, showing portions thereof in phantom outline;

FIG. 18A is a side sectional view of a catheter device usable in a further preferred method according to the invention;

FIG. 18B is a bottom view of the catheter of FIG. 18A;

FIG. 19A is a side sectional view of a dissolvable capsule/catheter device usable in another preferred method according to the invention;

FIG. 19B is a view, similar to FIG. 19A, of another dissolvable capsule/catheter device usable in further preferred method according to the invention; and

FIG. 19C is a view, similar to FIG. 19A, of yet another dissolvable capsule/catheter device usable in still further preferred method according to the invention.

### DETAILED DESCRIPTION OF SEVERAL PREFERRED EMBODIMENTS

Until quite recently, prior art apparatus and methods of treatment for patients infected with viruses which the body could not defeat with its own immune system, only delayed death. The methodologies of the present invention provide a means for destroying viruses, such as, for example, the HIV virus, and also lend themselves to treating a wide range of blood-borne pathogens, including bacteria, viral, fungal and vector induced infections. The objects are achieved in the present invention, inter alia, by way of modifications made to several pre-existing therapeutic delivery systems which have the effect of achieving significant improvement in the in situ delivery of therapeutic metal ions, metal alloys, metal salts (and combinations thereof) to the body of an infected patient in a controlled manner.

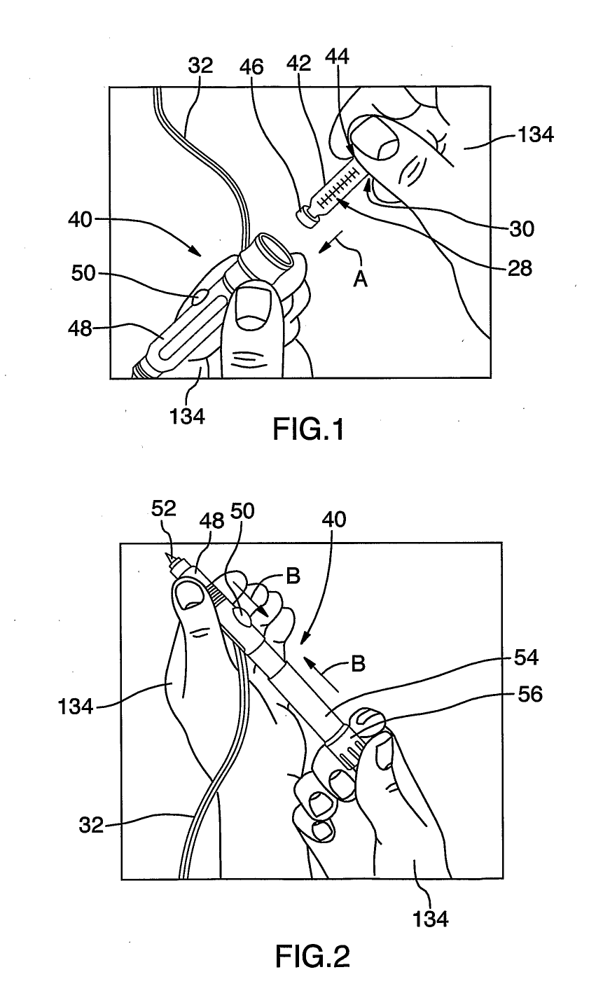

Several preferred modes and methods of delivering therapeutically efficacious doses of metal substances (including metal ions, salts, alloys and combinations thereof) to plants, animals and humans, both with and without electrical stimulation, are described hereinbelow and illustrated in FIGS. 1 through 19C.

Among others, the preferred metals and metal ions according to the invention may include therapeutic silver, gold and copper ions. Hereinbelow, a special emphasis may have been placed on methods of delivering silver ions which are produced in situ in therapeutically efficacious doses, but;the invention is not so limited, and extends to the therapeutic delivery of other metals, and metal ions, salts, alloys, and combinations thereof. It is therefore worthwhile to once again note, as discussed hereinabove, that the preferred metal substances according to the invention include silver, gold, copper, zinc, selenium, platinum, and their ions, alloys, salts, and combinations thereof, as well as other metals which may have been implicated as having a therapeutic value in association with different diseases and infections.

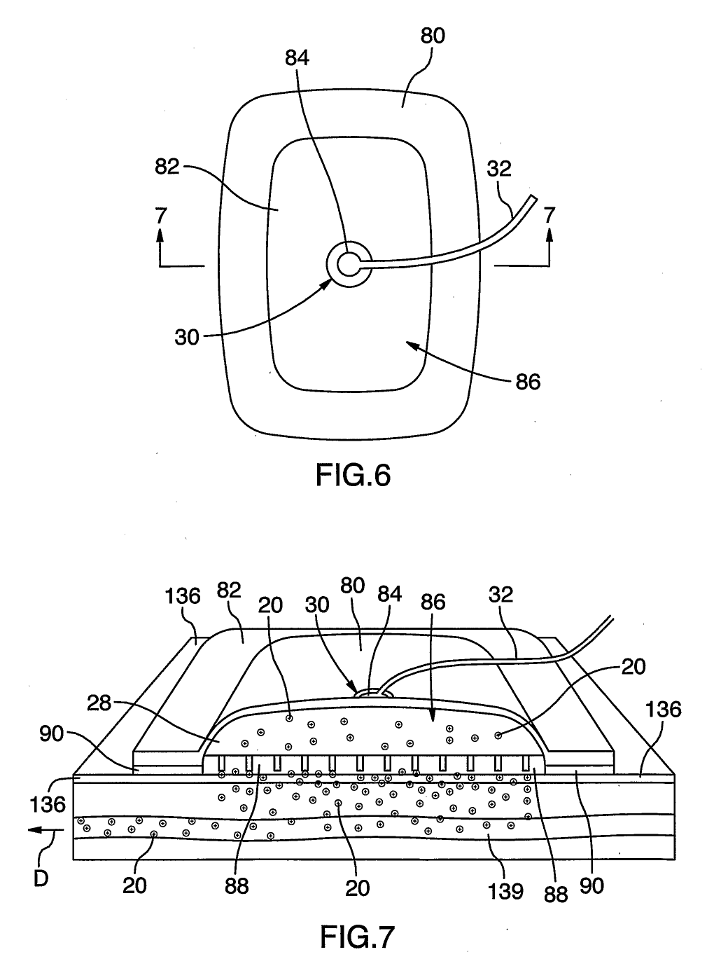

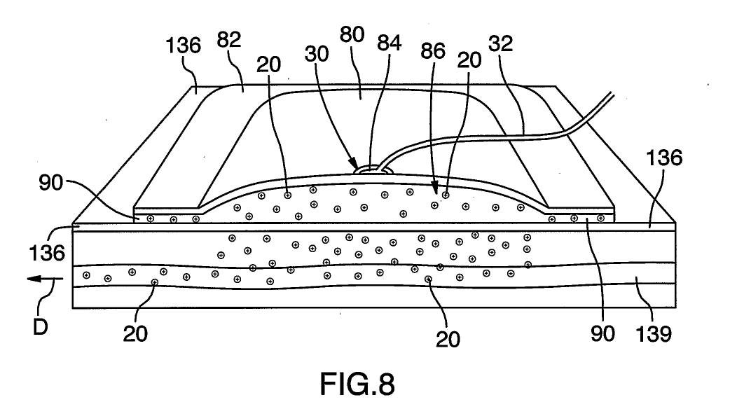

As aforesaid, a number of preferred modes and methods of delivery are described herein—only selected embodiments of which incorporate the use of a transdermal patch 80 (as shown in FIGS. 6 to 8). While transdermal patch methodologies will be discussed in greater detail hereinbelow, it is worthwhile to presently note that the application of transdermal patches 80 at particular desired sites on the skin 136 of an organism 130, as best seen in FIGS. 7 and 8, is generally thought, though not essential to the invention, to offer improved localization of delivery and/or generation of therapeutic metal ions. According to the invention, transdermal patches 80 may contain silver salts and, upon electrical stimulation, the penetration of silver into the epidermis may be facilitated, thus delivering in situ a therapeutic dose of silver ions. It is generally thought, though once again not essential to the invention, that the localization of the effect of transdermal patches 80 may allow an overall quantity of silver salts that are delivered to the organism 130 to be greatly reduced. As a result, any toxicity and/or other unwanted side effects which may generally be associated with systemic administration of such materials may be eliminated or greatly reduced.

Several new and specific applications fall within the scope of the present invention. A number of the embodiments described herein may be divided into two subgroups: (1) versions of the respective devices or procedures and improvements thereon which do not require electricity, and (2) versions of the respective devices or procedures and improvements thereon that do require electricity. According to the invention, therefore, delivery methods are disclosed which both do and do not involve the application and/or introduction of an electrical impulse to the organism 130.

In the electrical embodiments, an electrical current is typically introduced into the body 132 of the organism 130 substantially in the course of utilizing said delivery methodology. In the electrical embodiments that are further detailed hereinbelow, including those that relate to transdermal patches 80, the electrical stimulation or impulse can be introduced in a constant manner or it may be varied over time. Where the electrical current is varied over time, it may be intermittent and/or otherwise varied according to a preprogrammed schedule, including, for example, according to “on demand” schedules. Preferably, but not necessarily, and as further described hereinbelow, the electrical current may be a reversing electrical current.

As elsewhere described herein, there are a number of methods according to the invention which involve the production of metal ions using electrical charge, metal salts, metal alloys or combinations thereof. Appropriate electrical voltage for producing the metal ions may, in some embodiments, generally range between about 0.5 volts and about 3.0 volts, with about 1.5 volts being preferred. Appropriate electrical current for producing the metal ions may, in some embodiments, generally range between about 0.001 Amperes and about 0.01 Amperes, with about 0.01 Amperes being preferred. Accordingly, it may be appreciated that the power rating of some of the preferred embodiments, according to the invention, may be in the approximate order of about 0.015 watts.

As mentioned hereinabove, other modes and methods of delivery are discussed herein, and these may include syringe, auto-injector, and pricking device delivery methodologies (hereinafter alternately referred to as pin and/or pen prick methodologies), the use of contact stun guns, subcutaneous embedding, buccal embedding techniques (including placement and/or embedding of metal substances under the surface of the gums), aerosol inhaler techniques, ingestible dissolvable capsule methodologies, encapsulated bolus methodologies, and electrode catheterization methodologies.

As discussed hereinabove, and as shown in FIGS. 1 to 4, the delivery methodologies according to one series of embodiments of the invention may include syringe, auto-injector, and pricking device delivery methodologies. The epi-pen is an existing apparatus that has been previously used to deliver epinephrine to overcome anaphylaxis, thus the name ‘epi-pen’. It is an auto-injector used as a medical delivery system of a single dose of a particular drug. Similarly, “insulin pens” are known in the prior art. Most prior art auto-injector devices have been spring loaded syringes which, by design, have been easy to use and intended for use by the layperson. Auto-injector devices have heretofore been used to administer drugs through various different entry points on the body. Typical entry points have included the thigh and/or buttocks.

According to the invention, and instead of the delivery of epinephrine as in the prior art, a modified auto-injector device 40 is used to deliver metal ions, metal salts, alloys and combinations thereof, particularly silver salts, which may be substantially neutral in nature. According to these embodiments, and as best seen in FIG. 1, a therapeutically effective dose of the desired metal substance (e.g., silver), in a colloidal suspension 28 with a pharmaceutically acceptable carrier, is loaded into a dosage container 42 that is adapted to hold same. The dosage container 42 is then loaded (in the direction generally indicated by arrow “A” in FIG. 1) into a proximal housing portion 48 of the modified auto-injector device 40.

As best seen in FIG. 2, the proximal housing portion 48 is then assembled (in the directions generally indicated by arrows “B” in FIG. 2) with a distal housing portion 54 to assemble the auto-injector device 40.

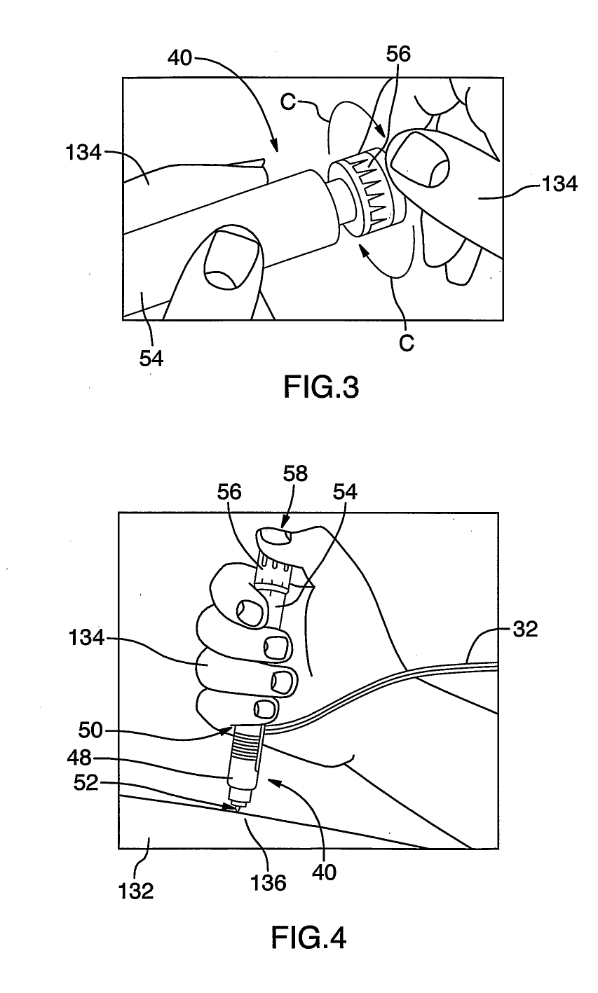

As shown in FIG. 3, the administrator (and/or patient organism 130 in the case of a human patient) may then preferably, but not necessarily, rotate a dosage dial 56 of the auto-injector 40 (as indicated generally by arrows “C” in FIG. 3) to select the desired therapeutic dosage level of the metal substance, as may preferably be ascertainable with reference to the species and body weight of the organism 130. Predetermined procedures may preferably, but not necessarily, outline specific dosage requirements (as well as specific time allotments while administering to the patient 130).

Thereafter, and as best seen in FIG. 4, the auto-injector device 40 may preferably be placed adjacent a target entry point on the organism 130. An injection button 58 thereof may be depressed to deliver the selected therapeutic dose of the metal substance, out of a proximal end portion 46 of the dosage chamber 42, and through a needle 52 of the auto-injector device 40 into the body 132.

The auto-injector delivery method that is shown in FIGS. 1 to 4, and discussed hereinabove, may preferably, but not necessarily, have equal application to both non-electrical and electrical embodiments alike.

This fact notwithstanding, however, in electrical embodiments of the auto-injector delivery method, and as best seen in FIG. 1, an electrode 30 is preferably formed substantially adjacent to a distal end portion 44 of the dosage container 42. In the electrical embodiments, the auto-injector device 40 is preferably, but not necessarily, also provided with a dosage charging button 50 (as shown in FIGS. 1 and 2).

As may be best appreciated from a consideration of FIG. 4, the administrator of the therapeutic dose may preferably, but not necessarily, use one of her hands 14 to depress the charging button 50 prior to depressing the injection button 58. When the charging button 50 is depressed, an electrical current may be introduced into the electrode 30—from an internal electric current source, such as, for example, a battery (not shown), and/or from an external electric current source, such as through a wire 32—so as to facilitate the delivery of the metal substance into the body 132 of the organism 130.

Speaking more generally, it is within the scope of the invention, according to the electrical auto-injector delivery methodologies, that the auto-injector device 40 may be provided in any configuration that is capable of introducing electrical current into the body 132 of the organism 130 in any manner whatsoever that facilitates delivery of the metal substance. The auto-injector device 40, containing, for example, metal salts, is allowed to “prick” at the desired entry site and, either with an AC or a DC power source, delivers freshly produced metal ions.

According to the invention, metal ions, metal salts, alloys and combinations thereof may be, in controlled doses, administered through the use of the modified auto-injector device 40 to treat various disease states. An example is the use of gold compounds in the treatment of arthritis. In further examples, silver salts may be used to treat eye infections, and selenium and zinc salts may be used in the control of macular degeneration and in the strengthening of the retina.

As discussed hereinabove, the delivery methodologies according to further embodiments of the invention may include buccal embedding techniques. Buccal embedding is a pre-existing technique and apparatus that may preferably use various medicinal regimes to implant a treatment under the surface of the gum. In the past, antibiotics have been implanted in the gums of patients to relieve infection.

According to the invention, and instead of the delivery of antibiotics as in the prior art, a modified buccal embedding technique may preferably provide for the introduction of various metal ions, metal salts, alloys, and/or combinations thereof, below the surface of the gums. According the buccal embedding techniques of the invention, the therapeutically effective dose of the metal substance, in a colloidal suspension with a pharmaceutically acceptable carrier, may preferably, but not necessarily, be loaded into a known syringe device. The syringe device may preferably then be used to inject the colloidal suspension containing the therapeutically effective dose of the metal substance below the surface of the gums in the buccal cavity of the body of the organism. In particularly preferred embodiments according to the invention, a pharmaceutically acceptable sealant is then applied substantially adjacent to the surface of the gums and substantially adjacent to a site of injection so as to impede escape of the colloidal suspension therethrough.

According to one embodiment of the inventive buccal embedding techniques, silver salts may be embedded under the gums and are engineered to deliver a constant or graded amount of silver ions. For many of the diseases of the mouth, such as, for example, carcinomas and ulcerations, a buccal implant of silver salts may be preferable to other prior art treatment regimes, since the therapeutic doses of silver ions can be delivered exactly where they are needed.

As discussed hereinabove, the delivery methodologies according to other embodiments of the invention may include contact dermal pressure devices. According to the invention, contact dermal pressure devices may preferably, but not necessarily, be equipped with silver salt (and/or other metal salt) tips. Extremely mild AC or DC power sources may be utilized according to these embodiments of the invention to effectively deliver therapeutic doses of silver (or other metal) ions to internal areas and/or organs that lay far below the immediate surface area. Although not essential to the invention, it is generally thought that electrical stimulation may induce an angiogenic response in cells.

As is also discussed hereinabove, the delivery methodologies according to still further embodiments of the invention may include subcutaneous embedding. Many present day injection methodologies use this mode of delivery, wherein known medicines and/or drugs have been physically injected so as to both systemically and/or locally administer therapeutic doses thereof. The prior art does not, however, disclose the subcutaneous embedding of metal substances as contemplated herein. According to the invention, therefore, subcutaneous embedding of a controlled release of the metal substance may be desirable, such as to exert some control over and/or ability to modify the length of exposure.

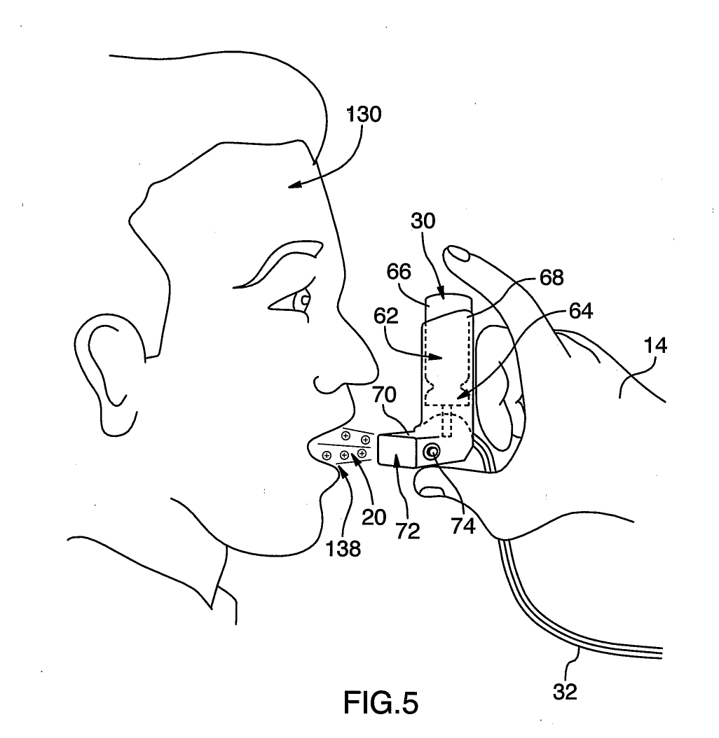

According to a further embodiment of the invention, and as shown in FIG. 5, the delivery methodologies may include aerosol inhaler techniques. Existing aerosol inhalers have heretofore, inter alia, been put to effective therapeutic use in the treatment of asthma. In the past, asthma medicaments have been loaded into the canisters of such existing aerosol inhalers to afford asthma sufferers some much needed respiratory relief at crucial times.

According to the invention, and as may be best appreciated from a consideration of FIG. 5, substantially particulate portions of the metal substance 20 may preferably each be respectively encapsulated within a pharmaceutically acceptable carrier (such as, for example, gelatin) and loaded, with a propellant, into a reservoir of a canister 62. The canister 62 is then preferably loaded into an upper portion 68 of a modified aerosol inhaler device 60. In use, the aerosol inhaler device 60 preferably sprays the therapeutic dose of the metal substance 20 (in the form of the particulate portions each encapsulated within their pharmaceutically acceptable carrier) out of a proximal end portion 64 of the canister 62, through a delivery aperture 72 that is formed in the lower portion 70 of the aerosol inhaler device 60, and into the mouth 138 and respiratory system of the organism 130.

The aerosol inhaler technique that is shown in FIG. 5, and discussed hereinabove, may preferably, but not necessarily, have equal application to both non-electrical and electrical embodiments alike.

This fact notwithstanding, however, in electrical embodiments of the aerosol inhaler technique, and as best seen in FIG. 5, an electrode 30 may be preferably formed substantially adjacent to a distal end portion 66 of the canister 62. In the electrical embodiments, the aerosol inhaler device 60 is preferably, but not necessarily, also provided with a dosage charging button 74 (as shown in FIG. 5). The administrator may preferably, but not necessarily, use one of his hands 14 to depress the charging button 74 prior to, or during, spray of the therapeutic dose of the metal substance 20 into the mouth 138 and respiratory system of the organism 130. When the charging button 74 is depressed, an electrical current may be introduced into the electrode 30—from an internal electric current source, such as, for example, a battery (not shown), and/or from an external electric current source, such as through a wire 32—so as to facilitate the delivery of the metal'substance into the mouth 138 and respiratory system of the organism 130.

Speaking more generally, it is within the scope of the invention, according to its electrical aerosol inhaler techniques, that the aerosol inhaler device 60 may be provided in any configuration that is capable of introducing an electrical current in a manner that facilitates spray delivery of the therapeutically effective dose of the metal substance from the aerosol inhaler device 60 into the respiratory system of the organism 130.

As discussed hereinabove, according to one of the preferred embodiments of the invention, and as shown in FIGS. 6 to 8, the delivery methodologies may include transdermal patch methodologies. In the prior art, transdermal patches have been used to deploy various medicinal regimes for the alleviation of infection and to promote healing. Such prior art transdermal patches and/or transdermal delivery systems have typically been adhesive patches that have heretofore been used to deliver controlled doses of conventional and known drugs.

In the prior art, transdermal patches have involved special membranes that control the rate of delivery of a liquid drug contained in an internal reservoir thereof to pass through the skin and into the bloodstream. Some conventional and known drugs have heretofore been combined with carrier substances, such as alcohol, so as to increase their ability to penetrate the skin. Conventional and known drugs previously administered using transdermal patches have included scopolamine for motion sickness, nicotine for smokers, estrogen for menopause and prevention of osteoarthritis after menopause, nitroglycerine for angina, and lidocaine to relieve pain of herpes zoster (shingles).

According to the present invention, however, and as best seen in FIG. 7, predetermined amounts (i.e., a therapeutically effective dose) of various metal ions, metal salts, alloys, or combinations thereof, may preferably be loaded onto a suitable undersurface or into a dosage chamber 86 of a modified transdermal patch 80.

The dosage chamber 86 of the transdermal patch 80 shown in FIG. 7 is a compartment that may preferably, but not necessarily, contain the metal substance 20 in a colloidal suspension 28 with a pharmaceutically acceptable carrier. The colloidal suspension 28 is preferably separated from an outer layer of skin 136 by a semi-permeable membrane 88. The membrane 88 forms the wall of the dosage chamber 86 in order to keep same compartmentalized and control the rate of release of the metal substance 20 therefrom. An adhesive layer 90 is provided, either as a continuous layer (not shown) between the membrane 88 and the skin 136, or preferably, and as generally depicted in FIG. 7, in a concentric configuration around the membrane 88. The adhesive layer 90 serves to adhere the various components of the patch 80 together and also to adhere the patch 80 to the skin 136. A backing layer 82, being the outermost layer of the transdermal patch 80 shown in FIGS. 6 and 7, preferably protects the colloidal suspension 28 of the metal substance 20 against the environment, prevents loss of the metal substance 20, and provides anchorage for the formulation.

FIG. 8 shows a different embodiment of the transdermal patch 80, according to the invention, that is provided in the form of single-layer metal-in-adhesive system. Similar reference numerals have been utilized throughout to designate and/or denote similar structures. The transdermal patch 80 shown in FIG. 8 is characterized by the inclusion of the metal substance 20 directly within the skin-contacting adhesive layer 90. In the transdermal patch 80 shown in FIG. 8, the adhesive layer 90 not only serves to affix the system to the skin 136, but also serves as the foundation for the therapeutic dose of the metal substance 20, and contains both the metal substance 20 and any excipients under its backing layer 82.

As shown in FIGS. 7 and 8, the transdermal patches 80,80 preferably release the metal substance 20 to pass through an outer layer of skin 136, from whence the metal substance 20 might possibly further travel into the organism's blood stream 139. Where it is desired that the metal substance 20 enter the blood stream 139, the direction of blood flow (as indicated generally by arrow “D” in FIGS. 7 and 8) may transport the metal substance 20 to the desired target.

The transdermal patch methodologies that are shown in FIGS. 6 to 8, and discussed hereinabove, may preferably, but not necessarily, have equal application to both non-electrical and electrical embodiments alike.

This fact notwithstanding, however, in electrical embodiments of the transdermal patch methodologies, and as shown in FIGS. 6 to 8, an electrode may preferably be formed from the metal substance 20 (or alternately from some other conducting material) and embedded in the transdermal patch 80. As may be best appreciated from consideration of FIGS. 7 and 8, an electrical current may preferably be introduced into the electrode 30, so as to facilitate delivery of the therapeutically effective dose of the metal substance 20 from the transdermal patch 80 into the organism's skin 136 and blood stream 139.

The electrical current may be introduced into the electrode 30 of the transdermal patch 80 either from an internal electric current source, such as, for example, a battery (not shown), and/or from an external electric current source, such as, for example, through a wire 32 which may be connected to an electrical contact 84 formed on an exterior surface of the backing layer 82 (as shown in FIGS. 6 to 8). Alternately, the electrical current may be introduced into the electrode 30 by contact with a stun gun type dermal press (not shown).

That is, in the electrical embodiments, transdermal patches 80 similar to those non-electrical embodiments described above may additionally and preferably, but not necessarily, incorporate an electrode 30 made of silver (or other metal, metal ions or alloys or combinations thereof), in the form of a prick or a prong, which may be activated by a stun gun type device. In this manner, a very low amount of electrical shock is transmitted to the organism's skin 136, but a sufficient amount to facilitate traversal of the metal ions across the epidermis and/or embedded area.

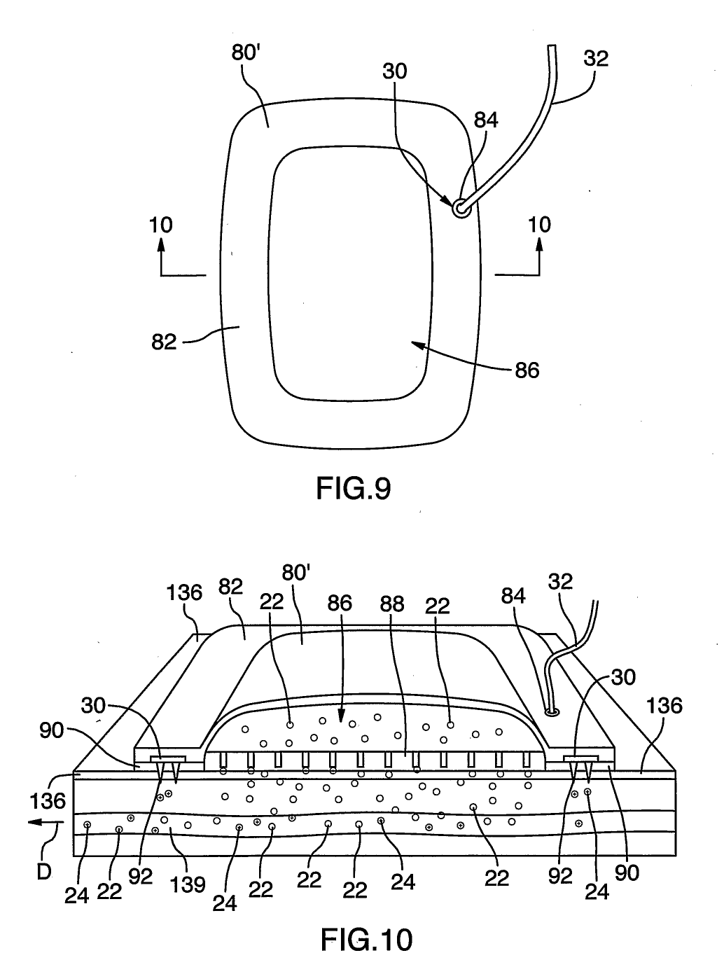

According to one embodiment of the invention, and as shown in FIGS. 9 and 10, the delivery methodologies may include needle transdermal patch, or “prickly” patch, methodologies. This modality is much like the transdermal patch methodologies described hereinabove, and shown in FIGS. 6 to 8, but is additionally and preferably provided with any number of small needle-like extensions, or needle members 92, on the underside of the patch 80′. Similar reference numerals have been utilized throughout to designate and/or denote similar structures.

As may be best appreciated from a consideration of FIG. 10, the electrode 30 may preferably be shaped in a concentric ring about the dosage chamber 86. According to this embodiment of the invention, the electrode 30 is preferably further shaped to define the needle members 92 that may preferably, but not necessarily be, formed from the metal substance 20. Preferably, at least one of the needle members 92 of the transdermal patch 80′ substantially penetrates the organism's outer layer of skin 136. As best seen in FIG. 10, the needle members 92 (whether made of silver or another metal substance) may preferably act as the electrodes 30 to deliver metal ions 24. Of course, other metals (i.e., other than silver) can be used to deliver the corresponding metal ions.

As with the other transdermal patches 80,80 discussed hereinabove, the underside of the needle transdermal patch 80′, according to the present invention, is saturated with metal ions 24, metal salts 22, alloys, or combinations thereof, and may or may not be activated using an external source of electric current, such as, for example, a wire 32 (as shown in FIGS. 9 and 10), or a stun gun (not shown) or an remotely controllable battery system (not shown). The introduction of the electric current preferably emits a low amount of electrical current, causing the metals ions 24, metal salts 22, alloys or combinations thereof to actually traverse the epidermis 136 and/or embedded area. Gold electrodes coupled with topical ointments may in fact augment the efficacy of both. The exact amounts of voltage, wattage and amperage may vary accordingly, but most studies place safe electrical stimulation at or close to about 1.0 volts at about 0.01 Amps, which would be a power rating of about 0.01 Watts. Reverse power modes may also be used here.

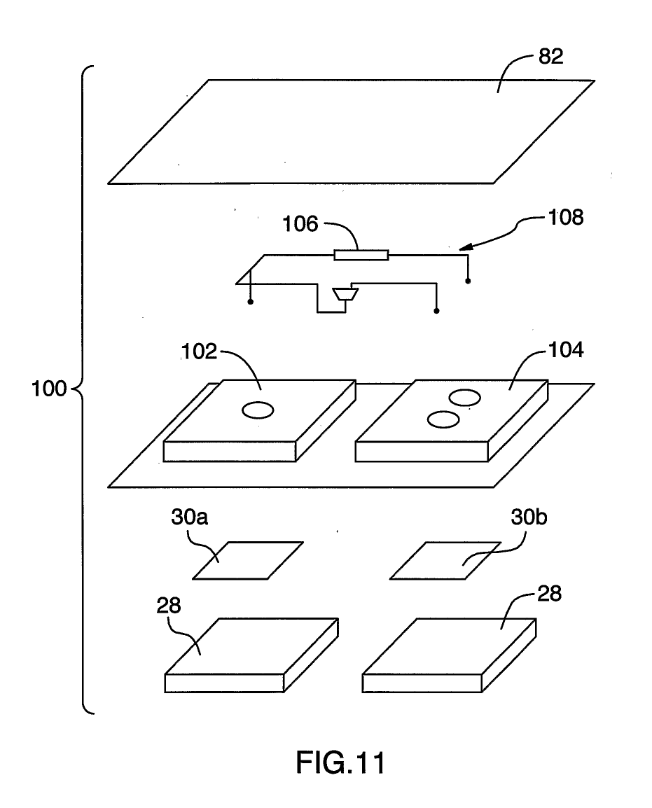

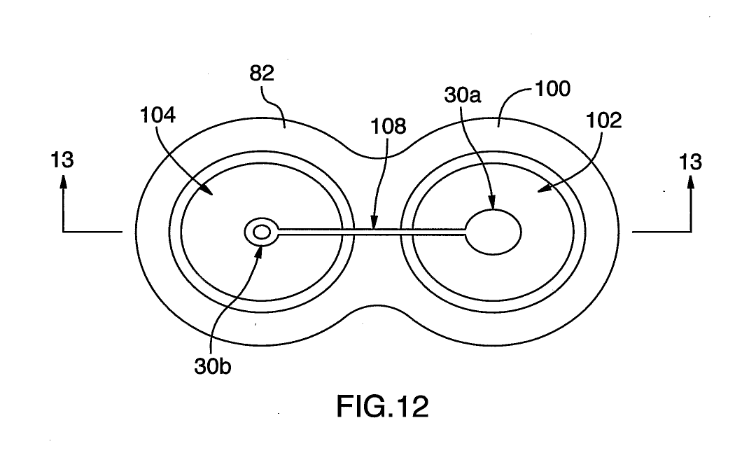

According to another embodiment of the invention, and as shown in FIGS. 11 to 14, the delivery methodologies may include encapsulated bolus methodologies. From the prior art, it may be appreciated that an encapsulated bolus acts and appears much like a prior art transdermal patch, but with the metal salts prepared on the undersurface of the patch presenting themselves as a large rounded area, or bolus, which is encapsulated or surrounded by an envelope. In FIGS. 12 to 14, and elsewhere, similar reference numerals have been utilized to designate and/or denote similar structures.

One prior art encapsulated bolus device that may be adapted for use with the present invention is disclosed in U.S. Pat. No. 6,775,570 that was issued to Joshi on Aug. 10, 2004—with the teachings of this patent being hereby incorporated herein by reference. As generally shown in FIG. 11, the Joshi patent discloses a low cost, accurate, single use, disposable, iontophoretic fluid delivery device, or encapsulated bolus device 100, having cationic and anionic chambers 30a, 30b separated by container structure and arranged to promote a flow of treatment ions into a body. The Joshi device desirably uses rugged mini-batteries to safely provide increased electromotive force to the ion transfer process in comparison to galvanic cells having electrolyte matched to a human body's electrolyte. Mini-batteries may be located in one or both cationic and anionic chambers 30a, 30b. One or more electric circuit components 106 may be arranged in a parallel circuit 108 to the body to provide enhanced efficacy of the device 100. For example, the electric circuit component 106 may be a shunt resistance that may be provided to control delivery of a beneficial agent in an amount over a time interval corresponding to any portion of a battery capacity—typically, between about 1 milliamps per minute and about 500 milliamps per minute, or more. The Joshi patent contemplates that substrates are to be located in the chambers and adapted to hold electrolyte or treatment drugs. The substrates in the Joshi patent may be electrically conductive to resist polarization of the chemicals near a conducting terminal. The cationic and anionic chambers 30a, 30b may be made having different sizes and/or shapes to facilitate placement of treatment drugs into the correct chamber, according to the Joshi patent.

The present invention preferably utilizes metal ions, metal salts, alloys, or combinations thereof, in association with modified versions of the encapsulated bolus device 100 disclosed by the Joshi patent.

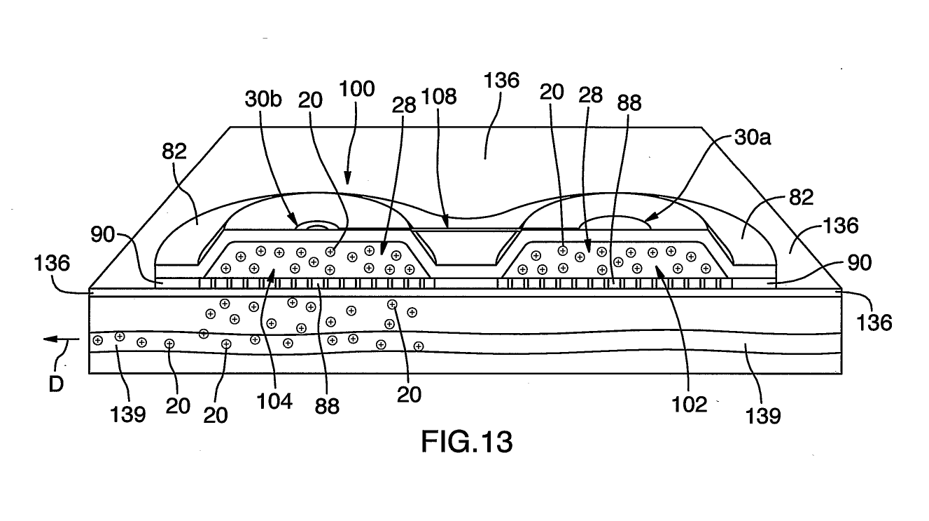

With specific reference to FIGS. 12 to 14, therefore, it will be appreciated that in the encapsulated bolus methodologies according to the invention, a cathode 30a is embedded substantially adjacent to a cationic chamber 102 that is defined within an encapsulated bolus device 100 (as described initially with reference to FIG. 13). Similarly, an anode 30b is embedded substantially adjacent to a separate anionic chamber 104 that is further defined within the encapsulated bolus device 100. A therapeutically effective dose of the metal substance 20, in a colloidal suspension with a pharmaceutically acceptable carrier, is embedded in at least one of the anionic chamber 104 and the cationic chamber 102 (and preferably, but not necessarily, in both).

When the electrical current is initially introduced into at least one of the anode 30b and the cathode 30a, the encapsulated bolus device 100 facilitates delivery of the therapeutically effective dose of the metal substance 20 from the anionic chamber 104, across the organism's outer layer of skin 136. More preferably, but not necessarily, the metal substance 20 may traverse, still further, into the organism's blood stream 139, whereupon it may be carried by the blood flow “D” to a desired target.

Preferably, in the novel and inventive encapsulated bolus methodologies that are disclosed according to the present invention, the electrical current may be substantially varied over time, and is a reversing electrical current.

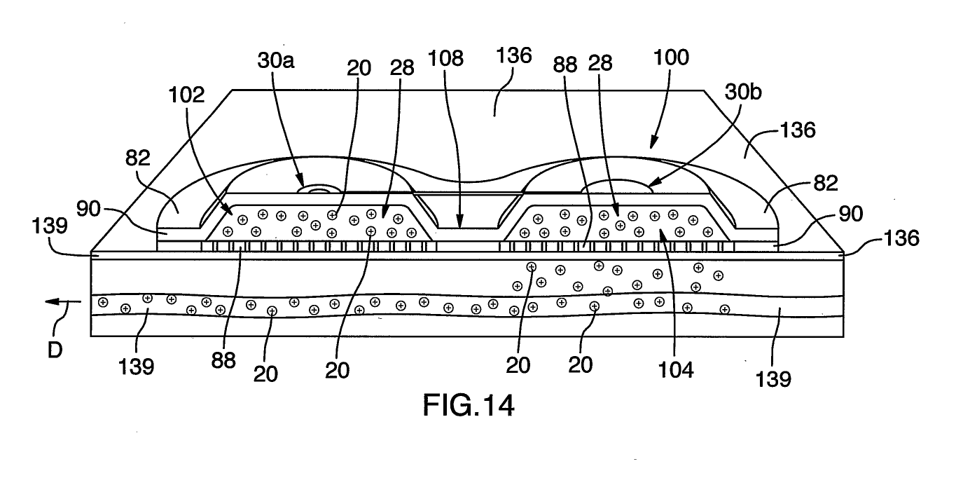

As such, and upon initially reversing the electrical current, and as shown in FIG. 14, the previous positions of the anode 30b and cathode 30a which were described above and shown in FIG. 13 may likewise be preferably, but not necessarily, effectively reversed (as shown in FIG. 14).

With the electrical current so reversed, the encapsulated bolus device 100 may preferably facilitate delivery of the therapeutically effective dose of the metal substance 20 from the anionic chamber 104 as shown in FIG. 14 (nee cationic chamber 102 as shown in FIG. 13), across the organism's outer layer of skin 136. Preferably, the metal substance 20 may traverse, still further, into the organism's blood stream 139, whereupon it may likewise be carried by the blood flow “D” to a desired target.

The electrical current may be reversed a further number of times, until substantially the entire therapeutic dose of the metal substance 20 has been transferred across the outer layer of the organism's skin 136.

As with the transdermal patches 80, 80′ described hereinabove, a prick or a prong (not shown) may be attached to the prior art Joshi structure, which may similarly be activated by a stun gun type device, thus giving a very low amount of electrical shock, and facilitating the traversal of the metal ions 20 into the epidermis 136 and/or the embedded area. That is, according to the invention, the electrical current may be introduced into at least one of the anode 30b and the cathode 30a of the encapsulated bolus device by contact with a stun gun type dermal press (not shown), or by one or more batteries located substantially adjacent to one or more of the cationic chamber 102 and the anionic chamber 104. As with the Joshi device, an electric circuit shunt resisting component may be arranged in a parallel circuit 108 to the skin 136, such that a predetermined amount of the electrical current may be introduced to the skin 136 over a predetermined time interval. As described hereinabove with reference to the Joshi patent, substantially between about 1 milliamp per minute and about 500 milliamps per minute may be introduced to the organism.

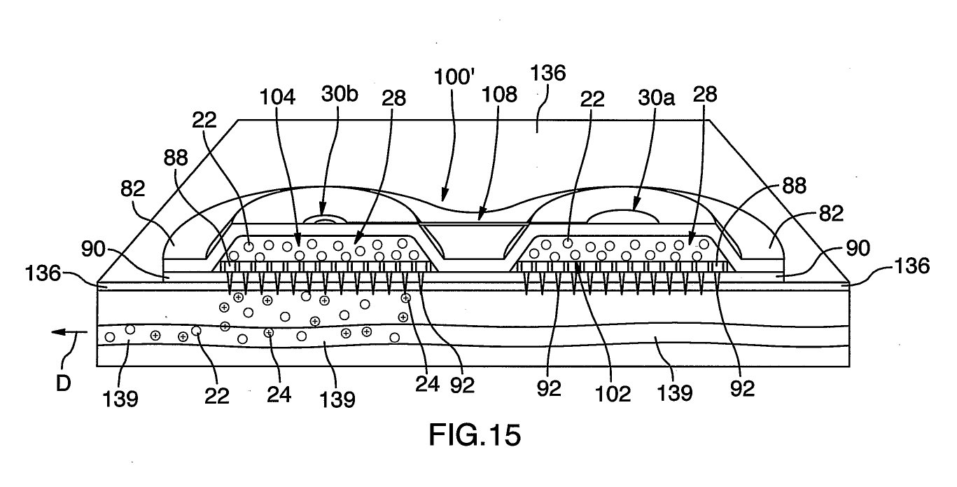

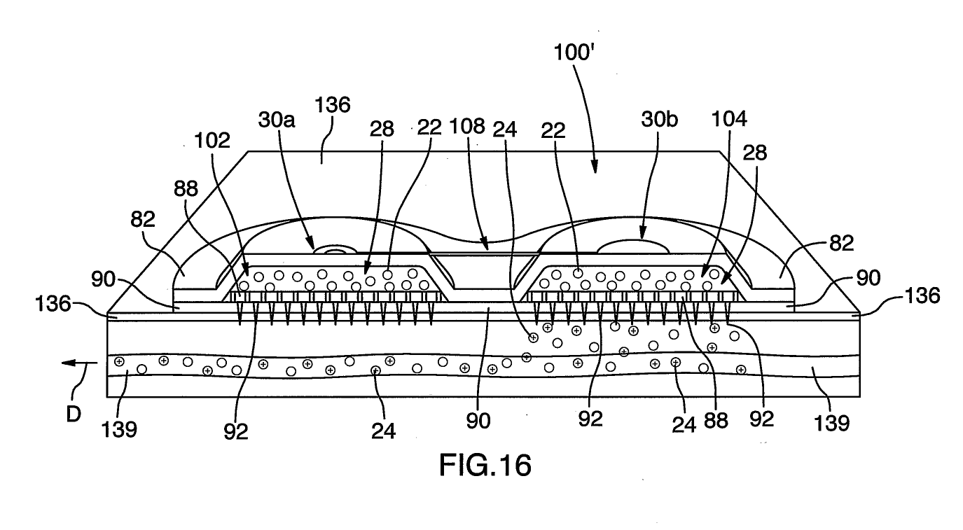

According to a further embodiment of the invention, and as shown in FIGS. 15 and 16, the delivery methodologies may include needle, or “prickly”, encapsulated bolus methodologies. These modalities share much in common with both the encapsulated bolus methodologies and the prickly patch methodologies, both of which are described hereinabove. Similar reference numerals have been utilized throughout to designate and/or denote similar structures. As shown in FIGS. 15 and 16, the prickly encapsulated bolus methodologies are additionally and preferably provided with any number of small needle-like extensions, or needle members 92, on the underside of a prickly encapsulated bolus device 100′. The needle members 92 preferably, but do not necessarily, extend from the anode 30b and cathode 30a of the prickly encapsulated bolus device 100′. Though a relatively minor difference, it is worthwhile to note that the needle members 92 of the prickly encapsulated bolus device 100′ shown in FIGS. 15 and 16 are depicted as having been provided within a substantially continuous adhesive layer 90 that underlies both the membranes 88 and the backing layer 82.

The needle members 92 may preferably, but not necessarily, be formed from the metal substance 20. Preferably, at least one of the needle members 92 of the prickly encapsulated bolus device 100′ substantially penetrates the organism's outer layer of skin 136.

When the electrical current is initially introduced into at least one of the anode 30b and the cathode 30a, the prickly encapsulated bolus device 100′ facilitates delivery of a therapeutically effective dose of metal salts 22 from the anionic chamber 104, along with metal ions 24 from the needle members adjacent the anionic chamber 104, across the organism's outer layer of skin 136. More preferably, but not necessarily, the metal salts 22 and metal ions 24 may traverse, still further, into the organism's blood stream 139, whereupon they may be carried by the blood flow “D” to a desired target.

Preferably, in the novel and inventive prickly encapsulated bolus methodologies that are disclosed according to the present invention, the electrical current may be substantially varied over time, and is a reversing electrical current.

As such, and upon initially reversing the electrical current, and as shown in FIG. 16, the previous positions of the anode 30b and cathode 30a which were described above and shown in FIG. 15 may likewise be preferably, but not necessarily, effectively reversed (as shown in FIG. 16).

With the electrical current so reversed, the prickly ecapsulated bolus device 100′ may preferably facilitate delivery of the therapeutically effective dose of the metal salts 22 and/or metal ions 24 from, and/or from substantially adjacent to, the anionic chamber 104 as shown in FIG. 16 (nee cationic chamber 102 as shown in FIG. 15), across the organism's outer layer of skin 136. Preferably, the metal salts 22 and metal ions 24 may traverse, still further, into the organism's blood stream 139, whereupon they may likewise be carried by the blood flow “D” to a desired target.

The electrical current may be reversed a further number of times, until substantially the entire therapeutic dose of the metal substance 20 has been transferred from the prickly encapsulated bolus device 100′ across the outer layer of the organism's skin 136.

Transdermal patch and/or encapsulated bolus systems may typically, but not necessarily, involve the addition and/or use of an enhancer or enhancing process—a mechanism or process to increase the permeability of the skin—and, in some of instances, also a mechanism to time the delivery and/or create bolus dosing. There are a number of enhancers and enhancing processes which might be used to facilitate drug delivery, possibly including each of the following: iontophoresis, ultrasound, chemicals including gels, microneedles, sonophoresis, lasers, and electroporatic methods.

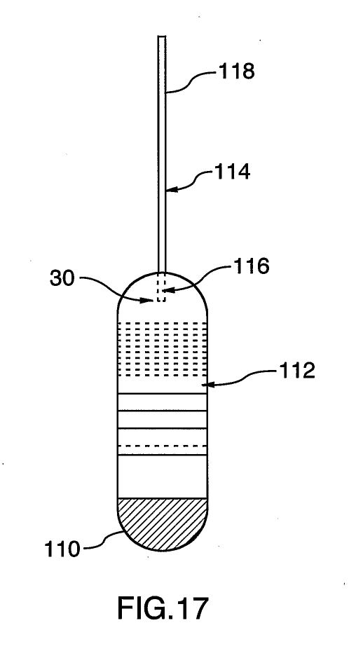

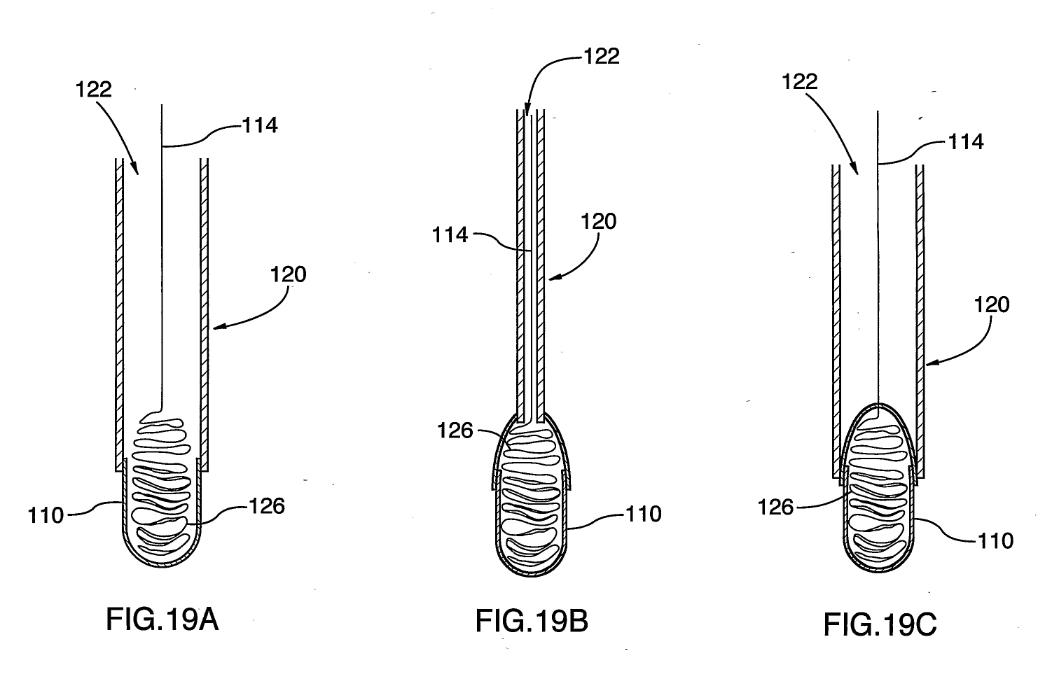

According to a yet further embodiment of the invention, and as shown in FIGS. 17 and FIGS. 19A to 19C, the delivery methodologies may include ingestible dissolvable capsule methodologies. Dissolvable gelatin capsules are known in the prior art. Such gelatin capsules may also have been known to be used in conjunction with a gastrointestinal tube guide and stiffener. One particularly useful dissolvable device of this type was originally created for obtaining samples of certain gastrointestinal pathogens (see U.S. Pat. No. 5,738,110 to Beal which is hereby incorporated herein by reference), and includes a gelatin pharmaceutical capsule containing a malleable drag material made of a mixture of beeswax and mineral oil.

According to the methodology of the present invention, and as best seen in FIG. 17, pharmaceutically acceptable (i.e., therapeutic) doses of the metal salts, metal ions, alloys, or combinations thereof, may be placed in the interior chamber 112 of a capsule 110 constructed generally as taught by Beal, as a colloidal suspension with suitable pharmaceutical diluents and/or carriers. The free end of the gelatin capsule 110 is preferably attached to a string member 114, which extends through a perforation in the capsule 110. According to the invention, the patient (not shown) may preferably hold the free end of the string member 114 and swallow the capsule 110. The gelatin capsule 110 dissolves in the stomach acid environment releasing the colloidal suspension.

That is, according to the invention, the therapeutically effective dose of the metal substance, in colloidal suspension with a pharmaceutically acceptable carrier, is loaded into the interior chamber 112 of the dissolvable capsule 110. The dissolvable capsule is introduced into at least one of the windpipe (not shown) and the foodpipe (not shown) of the organism, such that upon dissolving of the capsule 110, the therapeutically effective dose of the metal substance may preferably be delivered into at least a respective one of the lungs and the stomach of the organism.

According to the invention, the gelatin capsule 110 derived from the aforementioned Beal device may be further modified to ensure that the string member 114 connected to the capsule 110 is electrically conductive, and has at its end portion 116 inside the capsule 110 a tiny electrode 30 tipped with the metal substance (e.g., silver or other metal ions, salts, alloys or combinations thereof, as discussed herein). The electrically conductive string member 114 is preferably coated with a biocompatible insulating material 118.

By applying a low voltage to the electrically conductive string member 114, according to the invention, a melting of the gelatin capsule 110 may be effected, the exposing the metal ion electrode 30, and thereafter producing a condition conducive to spraying of the metal ions from the electrode 30 into the stomach or lung of the patient for treatment of pathogens found therein. In the case of the lung, the string member 114 is inserted into the windpipe and further into the desired part of the lung for the spray release of the metal ions. A silver ion tipped electrode 30 may preferably, but not necessarily, be particularly advantageous in the treatment of pathogens in the stomach and lungs. The length and other dimensions of the string member 114 and electrode 30 will preferably, of course, be sufficiently small that they will not puncture or tear any tissues of the patient during the retrieval process (i.e. when removed by retraction of the electrically conductive string).

The methodologies shown in FIGS. 19A through 19C represent modifications of the gelatin capsule method described hereinabove with reference to FIG. 17 but, at least insofar as they are used in conjunction with a gastro-intestinal tube guide and stiffener (these structures being substantially analogous to a catheter 120, with its lumen 126 and first electrode 126), additionally involve catheter-like aspects which may be further appreciated from the discussion of electrode catheterization which is provided hereinbelow. Again, similar reference numerals have been utilized to designate and/or denote similar structures.

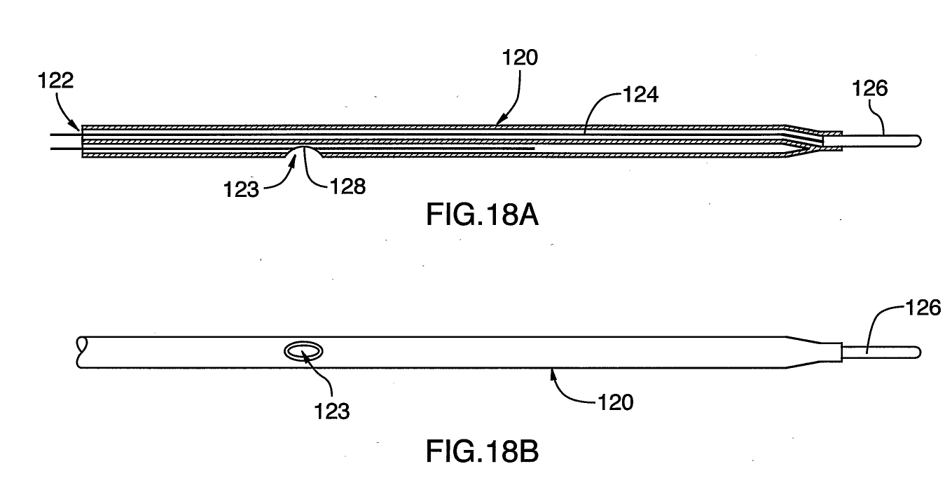

According to yet another embodiment of the invention, and as shown in FIGS. 18A and 18B, the delivery methodologies may include electrode catheterization methodologies. Delivery by electrode catheterization may be generally preferred where the therapeutic metal (e.g., silver) ions may be administered under professional care. Typically, electrode catheterization according to the invention is a treatment method whereby a silver wire, and/or other metal wire, may be maintained in the central blood circulation, with a low flow of electrical energy being used to generate silver and/or other metal ions and facilitate their distribution by the blood supply.

In the prior art, U.S. Pat. No. 6,066,489 issued to Fields and Burris on May 23, 2000 which patent is hereby incorporated herein by reference—disclosed a method and apparatus for destroying blood-borne pathogens utilizing a low intensity direct current to generate positive particles from various metals, which destroy viral pathogens. In the Fields and Burris patent, a first electrode comprised of a metal, such as silver, was disclosed as being inserted into a patient's venous system. Thereafter, a second electrode was placed on the patient's exterior in the vicinity of the first electrode. A low intensity direct current was then applied to the first metal electrode, thus releasing silver cations which were meant to bond to the offending virus, and resulting in its denaturation. According to the Fields and Burris patent, the first electrode is placed in the venous system of the infected patient via a catheter.

In the present invention, metal ions, metal salts, alloys or combinations thereof would be used as the materials of choice for patient treatment, along with a specific programmed regime as described hereinbelow.

According to the invention, therefore, and as shown in FIGS. 18A and 18B, an electrical conductor 124 is disposed within a lumen 122 of a catheter 120. A first electrode 126 extends out of the lumen 122 into the blood stream of the organism (not shown). The first electrode 126 is preferably in electrical communication with the electrical conductor 124. A second electrode 128 may be placed on the skin of the organism, or situated elsewhere in the blood stream. It will be appreciated that, upon introduction of electrical current into the electrical conductor 124 and the first electrode 126 from an external electric current source (not shown), the therapeutically effective dose of the metal substance is delivered into the body of said organism.

The first electrode 126 may preferably, but not necessarily, comprise a coating of the metal substance that is provided in substantially coating relation over a portion of the electrical conductor 124.

Possibly, but not necessarily, an electrolyte solution may be ionized and passed over the first electrode 126, such that the therapeutically effective dose of the metal substance is delivered from the ionized electrolyte into the body of said organism.

According to the invention, the external electric current source may be either an alternating current source or a direct current source.

Preferably, in the novel and inventive electrode catheterization methodologies that are disclosed according to the present invention, the electrical current may be substantially varied over time, however, and is most preferably, a reversing electrical current.

According to a specific embodiment of the invention, the electrode catheterization method may be carried out under hydration conditions, with a regimen adapted to substantially hydrate the organism being carried out before and during the electrode catheterization method.

This is a procedure utilizing a definite programmed regime of fluid hydration of the patient before the procedure, as well as during the procedure. Acceptable hydration before the procedure may include clean water taken orally for a designated period of time. During the procedure, the use of a sterile Ringer's solution, isotonic saline preparation, or like products, may be incorporated. It may be imperative to be hydrating the patient before, as well as, during the procedure for maximum results.

According to the electrode catheterization and hydration catheterization methods discussed hereinabove, the IV Giving Set and IV cannula or Abbocath or Intracath may be used, as may any other having a Y connector. A prepared sterile wire of the correct length, coated with a metal ion, metal salt, alloy, or any combination thereof, on the tip, is preferably inserted into the cannula (and/or catheter), so that the metal ion tip is at the distal end of the cannula.

An appropriate source of programmable electricity, or AC or DC battery, is needed to sustain and monitor the flow, amount of charge and the timing.

A separate coil of wire is placed around the IV bottle, producing an electromagnetic field when the controlled power source is applied, thus ionizing the electrolyte solution.

As the electrically charged electrolyte solution passes over the silver or appropriate metal ion tip, the silver or metal ion tip releases the ions located there. An ideal tip may be a combination silver, copper and platinum. Platinum may prevent corrosion on the tip surface, while copper assists in the conductivity. Reverse power methods may be applied here also.

In view of all of the various embodiments of the inventive method which are discussed hereinabove, it may be worthwhile to further discuss the manner in which the described electrical embodiments operate.

In use of the electrical embodiments, when a constant electrical current is passed through a conducting material, and then the current is allowed to pass through the metal to another conducting medium, such as a liquid solution, the interface between the two materials may have both electrical and dielectric properties.

The action of the electrical current at this interface is generally thought to ionize the metals with an electric charge that has an opposite polarity to the solution at the interface.

As a result of the attraction of opposite charges, and the repulsion of like charges, the ionized metal is generally thought to be repelled from the conductor and attracted into solution. The metal ions in solution may then be dispersed to the liquid medium by diffusion, electrical repulsion, and/or any flow of the liquid.

In solution, these charged metal ions are generally thought to be attracted to the active polarized sites of any pathogens which have opposite charge.

It should be appreciated that the discussion of the various uses which are provided herein may also preferably, but not necessarily, apply generally to other embodiments which are not illustrated, but which may fall within the scope of the invention.

In view of all of the various electrical embodiments of the inventive method which are discussed hereinabove, it may be worthwhile to further discuss the manner in which the described electrical embodiments differ from the prior art.

In the production of metal ions using electrical currents (with silver being the preferred ion in most of the therapeutic applications that are specifically described herein), it is normal in the course of these procedures for oxidized metals to be deposited at the interface between the conductor and the solution. This form of corrosion increases the dielectric constant at the interface, thereby impeding the ongoing production of metal ions. The corrosion is a function of the polarity of the applied electrical potential, and it is quite notably been determined that the deposition may preferably (but not necessarily) be reversed if, as contemplated according to the invention, the electrical field potential is reversed.

The time constants of the metal ion production and deposition of metal oxide are different. Some embodiments of the present invention take advantage of the differential time constants through the application of a reversal of the constant current flow according to an asymmetrical time schedule. In some of the preferred electrical embodiments, this schedule of current flow reversals is a reversing DC current which alternates according to an asymmetrical duty cycle of about 10 seconds in the positive direction, and then about 1 second in the reverse direction, continuing according to this schedule for a duration of about 15 minutes.

Alternately, the reversing electrical current may be a reversing DC current that alternates according to a substantially even duty cycle of about 1 second in the positive direction and about 1 second in the reverse direction, with the duty cycle continuing substantially as aforesaid for a duration of about 15 minutes.

It is perhaps worthwhile at this point to briefly discuss the potential use of the invention with specific reference to HIV. It is generally thought, though not essential to the invention, that when aggressive positive charges of metal ions, such as silver, are placed in the vicinity of a virus, the metal ions may be attracted to the negative polarity of the viral core. In the case of HIV, it is generally thought, though not essential to the invention, that such an attraction may lead to the formation of an ionic bond between the metal ion and the negative polarity of the HIV core. Although also not essential to the invention, it is generally thought that this ionic bond might lead to an exchange of an electron between the metal ion and viral proteins, and/or might lead to the denaturization of either the viral proteins or the breaking of the bonds in the virus RNA, thereby killing the virus. Once the virus is killed, it may be flushed from the blood by the patient's kidneys.

As discussed hereinabove, silver ion therapy may have particular application both in the treatment of HIV/AIDS and septicemia. It is generally thought, though not essential to the invention, that silver ions are produced in enormous quantities when an electric current is passed through silver metal in saline or other conducting solution, including blood. In fact, the amount of current needed is thought to be much lower than that which might be needed to affect the heart muscle. Silver ions are positively charged while viruses and bacteria may generally be thought to have a weak negative charge. It is thus likely that there is a mutual attraction between the ions and the viral and bacterial organisms, which may ultimately result the latter being inactivated by the ions.

It is worthwhile to further note that the half-life of the ionic form of silver in the blood may be calculated to be in the approximate order of about 7.8 seconds—a period which may be sufficient to give such silver ions enough time to react with many virions, particularly if delivery can be directly effected to a large blood vessel, such as the superior vena cava, which empties directly into the heart with blood from many different veins. In such a circumstance, with the blood entering the heart then being pumped immediately to the lungs or another target organ, the silver ions may still be active when they reach the target.

Notably, both cats and certain primates have been shown to be infected with fatal diseases that are caused by retroviruses similar to HIV. The feline immunodeficiency virus (FIV) appears to be transmitted to other cats through the saliva, such as, for example, through biting. Electrode catheterization treatment of FIV-infected cats with silver ions might be performed.

In humans, similar silver electrode catheterization therapies may be attempted to be applied to the cases of dying AIDS patients. In such circumstances, patients may be initially treated for 12 minutes at 2.5 microamps, and subsequently for 72 hours at 125 microamps. These trials may, but will not necessarily, result in precipitous drops in viral loads.

Catheters utilizable in such trials might be implanted in the subclavian vein (behind the clavicle), extending to the right auricle of heart, with the electrode protruding from the end of the catheter. One contemplated problem with such trials, however, may prove to be that the electrodes may be subject to relatively rapid oxidization, with a significant drop and/or cessation in the production of ions, within about 5 days. In such a circumstance, the silver electrodes would be required to be removed and replaced at frequent intervals, thus potentially reducing the effectiveness of any such treatment program.

### An Exemplary Test Procedure and Results

In one test performed according to the invention, infection studies were carried out in a Level III facility in vitro using HIV-infected and noninfected cells. To establish infection, a viral dose of MOI 0.5 was added to a culture flask. The supernatants of control and test flasks were tested for the presence of the p24 antigen. Cells were then incubated in the presence and absence of silver ions produced by passing current through a sterile silver electrode present in the culture flask. Current was applied for 0, 1, 5, 10 and 30 minutes after incubation of virus with the cells. Cells were then be maintained in culture for 7 days and the concentration of p24 antigen in the supernatant was determined.

The cells used for such studies were CEM-SS cultured human lymphoblasts to allow detection and monitoring of HIV-induced synceytium. Cells were grown in enriched RPMI medium (20% fetal calf serum; FCS) and maintained in a 5% CO₂ enriched tissue culture incubator at 37 degrees Celsius. Infection was conducted at a Multiplicity Dose of 0.5 using HIV_{IIIB} prepared from previously infected CEM-SS stock suspensions.

In this test performed according to the invention, a silver catheter was introduced by drill into the tissue culture flask, sealed and the flask was then gas-sterilized. Silver ions were generated by connecting the silver electrode to a purpose-built device designed by one of the investigators (RE) powered by a 9 volt battery and designed to generate a steady current of 1.0 milliamp.

Cell viability was determined using trypan blue dye exclusion. The HIV p24 antigen concentrations in the culture supernatants were determined using an antigen capture assay.

Each experiment was performed in triplicate and repeated three times. Differences between cells exposed and unexposed to silver ions were compared by analysis of variance.

This initial testing performed according to the invention demonstrated that a current of 1.0 milliamp applied through the silver electrode did not lead to a decline in viability of cultured non-infected human cells, and so this electrical current level was considered benevolent and was used as the standard level in the subsequent tests.

Even prolonged exposure of the non-infected human CEM-SS cells to the charged silver ions (for 10 minutes) was not associated with any significant decrease in viability (see Table 1).

TABLE 1
Cell Viability
Effect of Silver Ions on Normal vs. Infected
Zero minutes: (no treatment)

| | |
|---|---|
| Uninfected cells: | 100% |
| HIV-Infected Cells: | 20% |

One minute treatment:

| | |
|---|---|
| Uninfected cells: | 99% |
| HIV-Infected Cells: | 37% |

Ten minute treatment:

| | |
|---|---|
| Uninfected cells: | 97.5% |
| HIV-Infected Cells: | 54% |

As expected, in the absence of any applied current, cultures of CEM-SS infected with HIV_{IIIB} exhibited significant HIV-mediated syncytium formation and cell death. In contrast, cell viability was significantly increased and cytopathic effects of HIV infection were decreased following application of silver electrode current of 1.0 mA to the HIV pre-exposed cells. The beneficial effects of the current application were most marked following 10 minutes of silver ion exposure (Table 2), with a greater than 25% increase in cell viability for the infected cells, compared to similar cells not subjected to the silver ion treatment.

TABLE 2
Cell Viability 7 days post exposure
Effect of Treatment Time on HIV-Infected Cells suggests 10
minutes is optimal
Zero minutes: (no treatment)

| | |
|---|---|
| HIV-Infected Cells: | 20% |

One minute treatment:

| | |
|---|---|
| HIV-Infected Cells: | 37% alive |

Five minute treatment:

| | |
|---|---|
| HIV-Infected Cells: | 45% alive |

Ten minute treatment:

| | |
|---|---|
| HIV-Infected Cells: | 54% alive |

Thirty minute treatment:

| | |
|---|---|
| HIV-Infected Cells: | 28% alive |

These findings were corroborated by the quantitation of p24 antigen, a reliable quantitative indicator of HIV infection, in the culture supernatants of the HIV exposed cells. Supernatant analysis also revealed a marked decline in p24 antigen quantities associated with increasing times of exposure to 1.0 mA silver electrode current (see Table 1 hereinabove). After 10 minutes of this current flow, there was a decline in p24 antigen. Incubation of cells with silver ions at this current was not associated with a decrease in viability (see Table 1 hereinabove) among non-infected cells. In contrast, there was a decline in viability of HIV-infected cells, which appeared to improve with time (see Table 2 hereinabove).

In contrast, there was a marked decline in p24 antigen concentration associated with exposure of HIV pre-exposed human cell cultures to 1.0 mA of current through the silver electrode (see Table 1 hereinabove). Over thirty minutes, there was a 56% decline in the concentration of p24 antigen in the HIV-exposed cultures (p<0.05).

Many existing therapies for HIV infection have involved the use of anti-retroviral drugs, which although effective against HIV, have been associated with numerous adverse effects. Unfortunately, and as aforesaid, a major emerging issue is viral resistance, with the virus becoming increasingly resistant to therapy over the long term. Thus, new anti-retroviral agents are under development. As well, a number of novel therapies for HIV infection and AIDS are being evaluated. To the knowledge of the inventors, the testing detailed hereinabove according to the invention is the first study to investigate the use of a metal ion, in this case silver, as an antiretroviral intervention.

Though not essential to the invention, this testing appears to demonstrate that incubation of cells with silver ions generated by low voltage current inhibits the ability of HIV-1 to infect cells. This result does not appear to be related to changes in viability of non-infected cells, although there appear to be short-term changes in viability among HIV-infected cells. It is particularly striking to note that this effect was demonstrated a week later, after an incubation with silver ions for only 30 minutes.

How silver ions exert their anti-infectious effects is not clear, not essential to the present invention, but there are several possibilities. It is possible that there is a reduction in the number of cells infected with the virus; alternately, it is possible that there is reduced viral replication in silver-exposed cells. A number of further possibilities may exist as well.

It is generally thought that there is, normally, a small amount of silver that can be detected in the blood, at about 100 parts per billion of silver. There are several potential targets for increased concentrations of silver ions, including sulfhydryl groups. It has been noted that silver ions target sulfhydryl groups of some bacterial proteins modulating their activity. Thus, it is possible that the metal ions in the testing detailed hereinabove may mediate a similar effect and result in the inactivation of proteins critical for viral replication and pathogenesis such as HIV reverse transcriptase.

Notably, until now, no significant side effects attributable to this therapy have been observed. Unlike other available HIV oral therapies, such as protease inhibitors used in combination with other drugs, silver ion therapy has thus far generally been tolerable and not overly toxic to patients. It is perhaps worthwhile to note, however, that the use of silver therapy is not completely risk free. There are well-described syndromes of silver toxicity, primarily associated with long-term use of high doses of silver, which are generally outside the scope of the present invention. This is associated with slate-grey discolouration of the skin, but despite this, adverse effects on other organs have rarely been demonstrated.

As well, it is of interest to note that there appears to be an initial increase in the p24 concentration associated with silver ion incubation for five minutes, with a subsequent decline with time. It is possible that the initial exposure may be associated with some degree of cellular activation, which would enhance the ability of HIV-1 to infect cells.

Further studies, within the routine ken and competence of persons of ordinary skill in the art, to better define the optimal current and exposure time may be needed to optimize how this work may be extended in vivo. Accordingly, the optimal voltage, time of exposure and route of exposure may yet remain to be defined, but all fall within the scope of the present invention, even though considerable research may yet be required.

The testing according to the invention that is detailed hereinabove, however, would appear to have a role in demonstrating that exposure of cells to HIV in the presence of small amounts of silver ions may result in a marked reduction in HIV infection in the exposed cells. Given increasing problems with current antiretroviral therapy, these current results suggest that there may be a role for silver ion therapy as an otherwise unconventional approach which may ultimately be of value in the battle against HIV infection and AIDS.

Accordingly, it may be appreciated that silver ion therapy has the potential to have a profound effect on the progression of HIV infection, and may significantly improve the quality of life for patients with AIDS. It may likewise be appreciated that silver ion therapy has a significant potential to prove very effective in fighting other diseases and conditions, such as, for example, Hepatitis B and C and Parkinsons disease.

It may be further appreciated from,the above that the method described herein may be used to treat bacterial, viral, fungal, and/or vector-induced disease states, and to improve the health of humans, animals, and even plants (e.g., the banana plant). As aforesaid, delivery of the metal substance to the body of any particular organism may be adapted and/or varied according to the species and body weight of the organism.

With specific regard to animals, one or more of the methods described herein may be used to treat or preventatively treat hoof and mouth disease, leishmania, pig cholera, distemper, panleukopenia, panleukemia, heartworm disease, Johne's disease, feline immunodeficiency disease, and/or symptoms associated with any one or more of these conditions.

With specific regard to humans, one or more of the methods described herein may be used to treat or preventatively treat chagas, dengue, leishmania, encephalitis, rickettsia, candida, tuberculosis, pneumonia, septisemia, dysentary, polio, measles, chicken pox, small pox, mumps, ebola, malaria, eye infections, macular degeneration, retinal weakening, precursors to cancer, HPV, skin cancers, nasal pharyngeal cancer, breast cancer, prostate cancer, other carcinomas, diabetes, thyroid disorders, arthritis, transplant rejections, other autoimmune disease states, HIV, and/or symptoms associated with any one or more of these conditions.

The use of silver according to the invention may also be effective in treatment of burn victims, and against various other bacteria and viruses.

Of course, other modifications and alterations may be used in design and manufacture of embodiments according to the method of delivering therapeutic metal ions, salts and alloys, without departing from the spirit and scope of the invention, which is limited only by the accompanying claims.

1. A method for treating a disease state in the body of an organism, said method comprising a step of:
(a) delivering a therapeutically effective dose of a metal substance to the body of said organism using a delivery methodology selected from the group consisting of syringe, auto-injector, and pricking device delivery methodologies, buccal embedding techniques, transdermal patch methodologies, and aerosol inhaler techniques; wherein said metal substance is selected from the group consisting of silver, gold, copper, zinc, selenium, platinum, and their ions, alloys, salts, and combinations thereof.

2. A method according to claim 1, further comprising an additional step of:
(b) introducing an electrical current to the body of the organism substantially in the course of utilizing said delivery methodology.

3. A method according to claim 2 wherein, in step (b), said electrical current is substantially constant.

4. A method according to claim 2 wherein, in step (b), said electrical current is substantially varied over time.

5. A method according to claim 4, wherein said electrical current is substantially intermittent.

6. A method according to claim 4, wherein said electrical current is substantially varied over time substantially according to a preprogrammed schedule.

7. A method according to claim 4, wherein said electrical current is a reversing electrical current.

8. A method according to claim 1, wherein said delivery methodology is one of said syringe, auto-injector, and pricking device delivery methodologies; wherein before step (a), said therapeutically effective dose of said metal substance, in a colloidal suspension with a pharmaceutically acceptable carrier, is loaded into a dosage chamber of an auto-injector device; wherein in step (a), said auto-injector device is used to deliver said colloidal suspension containing said therapeutically effective dose of said metal substance into the body of said organism.

9. A method according to claim 2, wherein said delivery methodology is one of said syringe, auto-injector, and pricking device delivery methodologies; wherein before step (a), said therapeutically effective dose of said metal substance, in a colloidal suspension with a pharmaceutically acceptable carrier, is loaded into a dosage chamber of an auto-injector device, and an electrode is formed substantially adjacent to a distal end portion of said dosage chamber; wherein in step (b), said electrical current is introduced into said electrode so as to facilitate said delivery of said metal substance to the body of the organism.

10. A method according to claim 2, wherein said delivery methodology is one of said syringe, auto-injector, and pricking device delivery methodologies; wherein in step (a), an auto-injector device is used to deliver said therapeutically effective dose of said metal substance into the body of said organism; and wherein in step (b), said auto-injector device additionally introduces said electrical current so as to facilitate said delivery of said metal substance to the body of the organism.

11. A method according to claim 1, wherein said delivery methodology is one of said buccal embedding techniques; wherein before step (a), said therapeutically effective dose of said metal substance, in a colloidal suspension with a pharmaceutically acceptable carrier, is loaded into a syringe device; wherein in step (a), said syringe device is used to inject said colloidal suspension containing said therapeutically effective dose of said metal substance below the surface of the gums in the buccal cavity of the body of said organism.

12. A method according to claim 11, wherein after step (a), instep (a.1), a pharmaceutically acceptable sealant is applied substantially adjacent to the surface of the gums and substantially adjacent to a site of injection so as to impede escape of said colloidal suspension therethrough.

13. A method according to claim 1, wherein said delivery methodology is one of said transdermal patch methodologies; wherein before step (a), said therapeutically effective dose of said metal substance, in a colloidal suspension with a pharmaceutically acceptable carrier, is embedded in a transdermal patch; wherein in step (a), said transdermal patch releases said metal substance into the body of said organism.

14. A method according to claim 2, wherein said delivery methodology is one of said transdermal patch methodologies; wherein before step (a), an electrode formed from said metal substance is embedded in a transdermal patch; and wherein in steps (a) and (b), said electrical current is introduced into said electrode so as to facilitate said delivery of said therapeutically effective dose of said metal substance from said transdermal patch into the body of said organism.

15. A method according to claim 14, wherein said transdermal patch is a needle transdermal patch; and wherein before step (a), said electrode is shaped to define one or more needle members that are formed from said metal substance; wherein in step (a), at least one of said needle members of said transdermal patch substantially penetrates an outer layer of skin on the body of said organism.

16. A method according to claim 14, wherein in step (b), said electrical current is introduced into said electrode by contact with a stun gun type dermal press.

17. A method according to claim 1, wherein said delivery methodology is one of said aerosol inhaler techniques; wherein before step (a), substantially particulate portions of said metal substance are each respectively encapsulated within a pharmaceutically acceptable carrier and loaded with a propellant into a reservoir of a canister, with said canister then being loaded into an aerosol inhaler device; and wherein in step (a), said aerosol inhaler device sprays said therapeutic dose of said metal substance, in the form of said particulate portions encapsulated within said pharmaceutically acceptable carrier, into the respiratory system of said organism.

18. A method according to claim 2, wherein said delivery methodology is one of said aerosol inhaler techniques; wherein before step (a), substantially particulate portions of said metal substance are each respectively encapsulated within a pharmaceutically acceptable carrier and loaded with a propellant into a reservoir of a canister, with said canister then being loaded into an aerosol inhaler device, and an electrode is formed substantially adjacent to a distal end portion of said canister; and wherein in steps (a) and (b), said electrical current is introduced into said electrode so as to facilitate spray delivery of said therapeutically effective dose of said metal substance, in the form of said particulate portions encapsulated within said pharmaceutically acceptable carrier, from a proximal end portion of said canister of said aerosol inhaler device into the respiratory system of said organism.

19. A method according to claim 17, wherein in step (b), said pharmaceutically acceptable carrier is gelatin.

20. A method according to claim 9, wherein in step (b), said electrical current is introduced into said electrode from an external electric current source.

21. A method for treating a disease state in the body of an organism, said method comprising steps of:
(a) delivering a therapeutically effective dose of a metal substance to the body of said organism using a delivery methodology selected from the group consisting of ingestible dissolvable capsule methodologies, encapsulated bolus methodologies, and electrode catheterization methodologies; wherein said metal substance is selected from the group consisting of silver, gold, copper, zinc, selenium, platinum, and their ions, alloys, salts, and combinations thereof; and
(b) introducing an electrical current to the body of the organism substantially in the course of utilizing said delivery methodology; wherein said electrical current is substantially varied over time and is a reversing electrical current.

22. The method according to claim 21, wherein said delivery methodology is one of said ingestible dissolvable capsule methodologies; wherein before step (a), said therapeutically effective dose of said metal substance, in colloidal suspension with a pharmaceutically acceptable carrier, is loaded into an interior chamber of a dissolvable capsule; wherein in step (a), said dissolvable capsule is introduced into at least one of the windpipe and the foodpipe of the organism, such that upon dissolving of said capsule, said therapeutically effective dose of said metal substance is delivered into at least a respective one of the lungs and the stomach of said organism.

23. The method according to claim 22, wherein said dissolvable capsule is secured to an end portion of an electrically conductive string member encased in a biocompatible insulating material; wherein in step (b), after said gelatin capsule is introduced at least into the windpipe of the organism as aforesaid, said electrical current is introduced into said string member from an external electric current source, such as to charge said metal substance contained within said capsule.

24. The method according to claim 21, wherein said delivery methodology is one of said ingestible dissolvable capsule methodologies; wherein before step (a), a dissolvable capsule is secured to an end portion of an electrically conductive string member encased in a biocompatible insulating material, with said string member being in electrical communication with an electrode situated within said dissolvable capsule, with said electrode being at least coated with said metal substance; wherein in step (a), said dissolvable capsule is introduced into at least one of the windpipe and the foodpipe of the organism, and such that, thereafter, in step (b), said electrical current is introduced into said string member from an external electric current source, and from there into said electrode, such as to dissolve said capsule and deliver said therapeutically effective dose of said metal substance into at least a respective one of the lungs and the stomach of said organism.

25. A method according to claim 21, wherein said delivery methodology is one of said encapsulated bolus methodologies; wherein before step (a), a cathode is embedded substantially adjacent to a cationic chamber that is defined within an encapsulated bolus device, an anode is embedded substantially adjacent to a separate anionic chamber that is further defined within said encapsulated bolus device, and said therapeutically effective dose of said metal substance, in a colloidal suspension with a pharmaceutically acceptable carrier, is embedded in at least one of said anionic chamber and said cationic chamber; wherein in steps (a) and (b), said electrical current is introduced into at least one of said anode and said cathode so as to facilitate said delivery of said therapeutically effective dose of said metal substance from said ecapsulated bolus device into the body of said organism.

26. A method according to claim 25, wherein in step (b), said electrical current is introduced into said at least one of said anode and said cathode of said encapsulated bolus device by contact with a stun gun type dermal press.

27. A method according to claim 25, wherein in step (b), said electrical current is introduced into said at least one of said anode and said cathode of said encapsulated bolus device by one or more batteries located substantially adjacent to one or more of said cationic chamber and said anionic chamber.

28. A method according to claim 25, wherein before step (a), an electric circuit shunt resisting component is arranged in a parallel circuit to the body, such that in step (b), a predetermined amount of said electrical current is introduced to the body of said organism over a predetermined time interval.

29. A method according to claim 28, wherein in step (b), substantially between about 1 milliamp per minute and about 500 milliamps per minute are introduced to the body of said organism.

30. The method according to claim 21, wherein said delivery methodology is one of said electrode catheterization methodologies; wherein before step (a), an electrical conductor is disposed within a lumen of a catheter, a first electrode extends out of said lumen into the blood stream of said organism, with said first electrode being in electrical communication with said electrical conductor, and a second electrode is placed on the skin of the organism; and wherein in steps (a) and (b), said electrical current is introduced into said electrical conductor from an external electric current source, and from there into said first electrode, so as to deliver said therapeutically effective dose of said metal substance into the body of said organism.

31. The method according to claim 30, wherein said method is carried out under hydration conditions, with a regimen adapted to substantially hydrate the organism being carried out before and during said method.

32. The method according to claim 30, wherein said first electrode comprises a coating of said metal substance provided in substantially coating relation over a portion of said electrical conductor.

33. The method according to claim 30, wherein in step (b), said external electric current source is an alternating current source.

34. The method according to claim 30, wherein in step (b), said external electric current source is a direct current source.

35. The method according to claim 30, wherein before step (a), an electrolyte solution is ionized, and in at least one of steps (a) and (b), said electrolyte solution is passed over said first electrode, such that in step (b), said therapeutically effective dose of said metal substance is delivered into the body of said organism.

36. A method according to claim 21, wherein in step (b), an electric potential is substantially within the range of between about 0.5 and about 3.0 volts is introduced to the body of the organism, with said electrical current being substantially within the range of between about 0.001 amps and about 0.01 amps.

37. A method according to claim 36 wherein said electric potential is substantially in the order of about 1.0 volts, with said electrical current being substantially in the order of about 0.01 amps.

38. A method according to claim 36 wherein said electric potential is substantially in the order of about 1.5 volts, with said electrical current being substantially in the order of about 0.01 amps, and with a power rating substantially in the order of about 0.015 watts.

39. A method according to claim 21, wherein said metal substance comprises silver ions produced by said reversing electrical current, wherein said reversing electrical current is a reversing DC current that alternates according to a substantially even duty cycle of about 1 second in the positive direction and about 1 second in the reverse direction, with said duty cycle continuing substantially as aforesaid for a duration of about 15 minutes.

40. A method according to claim 21, wherein said metal substance comprises silver ions produced by said reversing electrical current, wherein said reversing electrical current is a reversing DC current that alternates according to an at least partially asymmetrical duty cycle of about 10 seconds in the positive direction and 1 second in reverse direction, with said duty cycle continuing substantially as aforesaid for a duration of about 15 minutes.

41. A method according to claim 21, wherein said reversing electrical current includes variations in cycle length.

42. A method according to claim 21, wherein said reversing electrical current includes variations in electrical current strength.

43. A method according to claim 21, wherein said reversing electrical current includes variations in electrical current duration.

44. The use of the method according to claim 21 to treat a bacterial disease state.

45. The use of the method according to claim 21 to treat a viral disease state.

46. The use of the method according to claim 21 to treat a fungal disease state.

47. The use of the method according to claim 21 to treat a vector-induced disease state.

48. The use of the method according to claim 21 to improve animal health.

49. The use according to claim 48, wherein delivery of said metal substance to the body of said organism is adapted to be varied according to a species of said organism and according to the body weight of said organism.

50. The use according to claim 48 for improving the health of an animal who has, or may develop, a condition selected from any one or more of: hoof and mouth disease, leishmania, pig cholera, distemper, panleukopenia, panleukemia, heartworm disease, Johne's disease, feline immunodeficiency disease, and symptoms associated therewith.

51. The use according to claim 48 to improve human health.

52. The use according to claim 51 for improving the health of a human who has, or may develop, a condition selected from any one or more of: chagas, dengue, leishmania, encephalitis, rickettsia, candida, tuberculosis, pneumonia, septisemia, dysentary, polio, measles, chicken pox, small pox, mumps, ebola, malaria, eye infections, macular degeneration, retinal weakening, and symptoms associated therewith.

53. The use according to claim 51 for improving the health of a human who has, or may develop, a condition selected from any one or more of: precursors to cancer, HPV, skin cancers, nasal pharyngeal cancer, breast cancer, prostate cancer, other carcinomas, and symptoms associated therewith.

54. The use according to claim 51 for improving the health of a human who has, or may develop, a condition selected from any one or more of: diabetes, thyroid disorders, arthritis, transplant rejections, other autoimmune disease states, and symptoms associated therewith.

55. The use according to claim 51 for improving the health of a human who has, or may develop, a condition selected from any one or more of HIV, and symptoms associated therewith.

56. The use of the method according to claim 21 to improve plant health.

57. The use according to claim 56 to improve the health of a banana plant.

58. An apparatus for delivering a metal substance to the body of an organism, wherein said apparatus comprises:
(a) a therapeutically effective dose of a metal substance, with said metal substance being selected from the group consisting of silver, gold, copper, zinc, selenium, platinum, and their ions, alloys, salts, and combinations thereof;
(b) a delivery apparatus that contains said metal substance and delivers said therapeutically effective dose of said metal substance to the body of the organism, with said delivery apparatus being selected from the group consisting of an auto-injector device, a transdermal patch, and an aerosol inhaler.

59. An apparatus according to claim 58, further comprising a power source that is adapted to introduce an electrical current to the body of the organism substantially in the course of delivering said therapeutically effective dose of said metal substance as aforesaid.

60. An apparatus according to claim 59 wherein said electrical current is maintained substantially constant by said power source.

61. An apparatus according to claim 59 wherein said power source substantially varies said electrical current over time.

62. An apparatus according to claim 61, wherein said electrical current introduced by said power source is substantially intermittent.

63. An apparatus according to claim 61, wherein said power source substantially varies said electrical current over time substantially according to a programmed schedule.

64. An apparatus according to claim 61, wherein said power source periodically reverses said electrical current according to a programmed schedule, such that said electrical current is a reversing electrical current.

65. An apparatus according to claim 58, further comprising a pharmaceutically acceptable carrier in a colloidal suspension with said therapeutically effective dose of said metal substance; wherein said delivery apparatus is said auto-injector device, with said auto-injector-device including a dosage chamber that contains said colloidal suspension.

66. An apparatus according to claim 59, further comprising a pharmaceutically acceptable carrier in a colloidal suspension with said therapeutically effective dose of said metal substance; wherein said delivery apparatus is said auto-injector device, with said auto-injector device including a dosage chamber and an electrode, with said colloidal suspension being contained within said dosage chamber, and with said electrode being positioned substantially adjacent to a distal end portion of said dosage chamber; and wherein said power source introduces said electrical current into said electrode so as to facilitate the aforesaid delivery of said therapeutically effective dose of said metal substance.

67. An apparatus according to claim 58, further comprising a pharmaceutically acceptable carrier in a colloidal suspension with said therapeutically effective dose of said metal substance; wherein said delivery apparatus is said transdermal patch, with said transdermal patch having said colloidal suspension releasably embedded therewithin.

68. An apparatus according to claim 59, wherein said delivery apparatus is said transdermal patch; wherein said transdermal patch has an electrode formed from said metal substance embedded therewithin; and wherein said power source introduces said electrical current into said electrode so as to facilitate the aforesaid delivery of said therapeutically effective dose of said metal substance.

69. An apparatus according to claim 68, wherein said transdermal patch is a needle transdermal patch; wherein said electrode is shaped to define one or more needle members that are formed from said metal substance; and wherein at least one of said needle members is adapted to substantially penetrate an outer layer of skin on the body of said organism.

70. An apparatus according to claim 58, wherein said delivery apparatus is said aerosol inhaler, with said aerosol inhaler comprising a canister that defines an internal reservoir therewithin; wherein said reservoir is loaded with a propellant and substantially particulate portions of said metal substance, with each of said particulate portions respectively being encapsulated within a pharmaceutically acceptable carrier; and wherein said aerosol inhaler is adapted to emit a spray of said particulate portions.

71. An apparatus according to claim 59, wherein said delivery apparatus is said aerosol inhaler, with said aerosol inhaler comprising a canister that defines an internal reservoir therewithin; wherein said reservoir is loaded with a propellant and substantially particulate portions of said metal substance, with each of said particulate portions respectively being encapsulated within a pharmaceutically acceptable carrier; and wherein said aerosol inhaler further comprises an electrode that is positioned substantially adjacent to a distal end portion of said canister; and wherein said power source introduces said electrical current into said electrode so as to facilitate spray delivery of said therapeutically effective dose of said metal substance, in the form of said particulate portions encapsulated within said pharmaceutically acceptable carrier, from a proximal end portion of said canister of said aerosol inhaler device.

72. An apparatus according to claim 70, wherein said pharmaceutically acceptable carrier is gelatin.

73. An apparatus according to claim 66, wherein said power source is situated substantially externally of said delivery apparatus.

74. An apparatus for delivering a metal substance to the body of an organism, wherein said apparatus comprises:
(a) a therapeutically effective dose of a metal substance, wherein said metal substance is selected from the group consisting of silver, gold, copper, zinc, selenium, platinum, and their ions, alloys, salts, and combinations thereof;
(b) a delivery apparatus that contains said metal substance and delivers a said therapeutically effective dose of said metal substance to the body of the organism, with said delivery apparatus being selected from the group consisting of an ingestible dissolvable capsule, an encapsulated bolus device, and an electrode catheter device; and
(c) a power source that is adapted to introduce an electrical current to the body of the organism substantially in the course of delivering said therapeutically effective dose of said metal substance as aforesaid, with said power source substantially varying said electrical current over time, and with said power source periodically reversing said electric current according to a programmed schedule.

75. An apparatus according to claim 74, further comprising a pharmaceutically acceptable carrier in a colloidal suspension with said therapeutically effective dose of said metal substance; wherein said delivery apparatus is said ingestible dissolvable capsule; wherein said dissolvable capsule defines an interior chamber therewithin, with said interior chamber containing said colloidal suspension, such that dissolution of said capsule facilitates the aforesaid delivery of said therapeutically effective dose of said metal substance.

76. An apparatus according to claim 75, wherein said dissolvable capsule is secured to an end portion of an electrically conductive string member, with said electrically conductive string member being encased in a biocompatible insulating material; wherein said power source is situated substantially externally of said dissolvable capsule; and wherein said power source introduces said electrical current into said string member such as to charge said metal substance contained within said capsule.

77. An apparatus according to claim 74, wherein said delivery apparatus is said ingestible dissolvable capsule, with said dissolvable capsule including an electrode that is situated therewithin; wherein said dissolvable capsule is secured to an end portion of an electrically conductive string member, with said electrically conductive string member being encased in a biocompatible insulating material, and with said string member being adapted to be in electrical communication with an said electrode; wherein said electrode is at least coated with said metal substance; and wherein said power source is situated substantially externally of said dissolvable capsule; and wherein said power source introduces said electrical current into said string member and from there into said electrode, such as to dissolve said capsule and deliver said therapeutically effective dose of said metal substance.

78. An apparatus according to claim 74, further comprising a pharmaceutically acceptable carrier in a colloidal suspension with said therapeutically effective dose of said metal substance; wherein said delivery apparatus is said encapsulated bolus device; with said encapsulated bolus device defining therewithin a cationic chamber and a separate anionic chamber; wherein said encapsulated bolus device comprises a cathode that is embedded substantially adjacent to said cationic chamber, an anode is embedded substantially adjacent to said separate anionic chamber, with said colloidal suspension being embedded in at least one of said anionic chamber and said cationic chamber; wherein said power source introduces said electrical current into at least one of said anode and said cathode so as to facilitate the aforesaid delivery of said therapeutically effective dose of said metal substance from said encapsulated bolus device.

79. An apparatus according to claim 78, wherein said power source comprises one or more batteries located substantially adjacent to one or more of said cationic chamber and said anionic chamber, with said batteries introducing said electrical current into said at least one of said anode and said cathode of said encapsulated bolus device as aforesaid.

80. An apparatus according to claim 78, further comprising an electric circuit shunt resisting component that is adapted for arrangement in a parallel circuit to the body; with said electric circuit shunt resisting component being adapted to introduce a predetermined amount of said electrical current to the body of said organism over a predetermined time interval.

81. An apparatus according to claim 80, wherein said electric circuit shunt resisting component is adapted to introduce substantially between about 1 milliamp per minute and about 500 milliamps per minute.

82. An apparatus according to claim 74, wherein said delivery apparatus is said electrode catheter device; wherein said electrode catheter device defines a lumen, with said electrode catheter device comprising an electrical conductor that is disposed within said lumen, and a first electrode that extends out of said lumen and is adapted to extend into the blood stream of said organism, with said first electrode being in electrical communication with said electrical conductor, and with said electrode catheter device further comprising a second electrode that is adapted to be placed on the skin of the organism; wherein said power source is situated substantially externally of said electrode catheter device; and wherein said power source introduces said electrical current into said electrical conductor, and from there into said first electrode, so as to facilitate the delivery of the aforesaid therapeutically effective dose of said metal substance.

83. An apparatus according to claim 82, further comprising a hydration means for substantially hydrating the organism before, and maintaining the hydration of the organism over the course of, delivering said therapeutically effective dose of said metal substance as aforesaid.

84. An apparatus according to claim 82, wherein said first electrode comprises a coating of said metal substance provided in substantially coating relation over a portion of said electrical conductor.

85. An apparatus according to claim 82, wherein said power source is an alternating current source.

86. An apparatus according to claim 82, wherein said power source is a direct current source.

87. An apparatus according to claim 82, further comprising an ionized electrolyte solution that is supplied in fluid communication over said first electrode such as to facilitate the aforesaid delivery of said therapeutically effective dose of said metal substance.

88. An apparatus according to claim 74, wherein the power source is adapted to provide an electric potential substantially within the range of between about 0.5 and about 3.0 volts to the body of the organism, with said electrical current being substantially within the range of between about 0.001 amps and about 0.01 amps.

89. An apparatus according to claim 88 wherein said electric potential is substantially in the order of about 1.0 volts, with said electrical current being substantially in the order of about 0.01 amps.

90. An apparatus according to claim 88 wherein said electric potential is substantially in the order of about 1.5 volts, with said electrical current being substantially in the order of about 0.01 amps, and with a power rating substantially in the order of about 0.015 watts.

91. An apparatus according to claim 74, wherein said metal substance comprises silver ions produced by said reversing electrical current, wherein said reversing electrical current is a reversing DC current that alternates according to a substantially even duty cycle of about 1 second in the positive direction and about 1 second in the reverse direction, with said duty cycle continuing substantially as aforesaid for a duration of about 15 minutes.

92. An apparatus according to claim 74, wherein said metal substance comprises silver ions produced by said reversing electrical current, wherein said reversing electrical current is a reversing DC current that alternates according to an at least partially asymmetrical duty cycle of about 10 seconds in the positive direction and 1 second in reverse direction, with said duty cycle continuing substantially as aforesaid for a duration of about 15 minutes.

93. An apparatus according to claim 74, wherein said reversing electrical current includes variations in cycle length.

94. An apparatus according to claim 74, wherein said reversing electrical current includes variations in electrical current strength.

95. An apparatus according to claim 74, wherein said reversing electrical current includes variations in electrical current duration.

96. The use of the apparatus according to claim 74 to treat a disease state in the body of the organism,

97. The use of the apparatus according to claim 96 to treat a bacterial disease state.

98. The use of the apparatus according to claim 96 to treat a viral disease state.

99. The use of the apparatus according to claim 96 to treat a fungal disease state.

100. The use of the apparatus according to claim 96 to treat a vector-induced disease state.

101. The use of the apparatus according to claim 74 to improve animal health.

102. The use according to claim 101, wherein delivery of said metal substance to the body of said organism is adapted to be varied according to a species of said organism and according to the body weight of said organism.

103. The use according to claim 101 for improving the health of an animal who has, or may develop, a condition selected from any one or more of: hoof and mouth disease, leishmania, pig cholera, distemper, panleukopenia, panleukemia, heartworm disease, Johne's disease, feline immunodeficiency disease, and symptoms associated therewith.

104. The use according to claim 101 to improve human health.

105. The use according to claim 104 for improving the health of a human who has, or may develop, a condition selected from any one or more of: Chagas, dengue, leishmania, encephalitis, rickettsia, candida, tuberculosis, pneumonia, septisemia, dysentary, polio, measles, chicken pox, small pox, mumps, ebola, malaria, eye infections, macular degeneration, retinal weakening, and symptoms associated therewith.

106. The use according to claim 104 for improving the health of a human who has, or may develop, a condition selected from any one or more of: precursors to cancer, HPV, skin cancers, nasal pharyngeal cancer, breast cancer, prostate cancer, other carcinomas, and symptoms associated therewith.

107. The use according to claim 104 for improving the health of a human who has, or may develop, a condition selected from any one or more of: diabetes, thyroid disorders, arthritis, transplant rejections, other autoimmune disease states, and symptoms associated therewith.

108. The use according to claim 104 for improving the health of a human who has, or may develop, a condition selected from any one or more of HIV, and symptoms associated therewith.

109. The use of the apparatus according to claim 74 to improve plant health.

110. The use according to claim 109 to improve the health of a banana plant.
